SEQUENCE LISTING (54 sequences)

SEQ ID NO: 1 - protein, 52 aa, Homo sapiens
  VARIANT 14: note = R14 in human WT PLN protein
MEKVQYLTRSAIRRASTIEMPQQARQKLQNLFINFCLILICLLLICIIVMLL

SEQ ID NO: 2 - protein, 51 aa, Homo sapiens
  VARIANT 14: note = position of R14del (deletion of R14) in human PLN R14del mutant protein
MEKVQYLTRSAIRASTIEMPQQARQKLQNLFINFCLILICLLLICIIVMLL

SEQ ID NO: 3 - DNA, 20 bp, synthetic construct
  source 1..20: mol_type = other DNA; note = Primer BAC sequencing #1
cacaccatgaccgtctcatt

SEQ ID NO: 4 - DNA, 22 bp, synthetic construct
  source 1..22: mol_type = other DNA; note = Primer BAC sequencing #2
tgctgaggaagatgaattagtg

SEQ ID NO: 5 - DNA, 20 bp, synthetic construct
  source 1..20: mol_type = other DNA; note = Primer BAC sequencing #3
cccaagtccagacacacaag

SEQ ID NO: 6 - DNA, 19 bp, synthetic construct
  source 1..19: mol_type = other DNA; note = Primer BAC sequencing #4
gacaatagcaggcatgctg

SEQ ID NO: 7 - DNA, 19 bp, synthetic construct
  source 1..19: mol_type = other DNA; note = Primer BAC sequencing #5
gtggatgtggaatgtgtgc

SEQ ID NO: 8 - DNA, 23 bp, synthetic construct
  source 1..23: mol_type = other DNA; note = Primer qPCR PLN forward
ggtttcacactgatagagggatt

SEQ ID NO: 9 - DNA, 22 bp, synthetic construct
  source 1..22: mol_type = other DNA; note = Primer qPCR PLN reverse
accagtaaagcagttgtctcat

SEQ ID NO: 10 - DNA, 23 bp, synthetic construct
  source 1..23: mol_type = other DNA; note = Primer qPCR USH forward
cttcttggagcagatgggataaa

SEQ ID NO: 11 - DNA, 23 bp, synthetic construct
  source 1..23: mol_type = other DNA; note = Primer qPCR USH reverse
gaatgcagatttctggtttccac

SEQ ID NO: 12 - DNA, 20 bp, synthetic construct
  source 1..20: mol_type = other DNA; note = Primer qPCR NG forward
gaggcttcacttgttaaggg

SEQ ID NO: 13 - DNA, 21 bp, synthetic construct
  source 1..21: mol_type = other DNA; note = Primer qPCR NG reverse
ctgagatcagggtagacatac

SEQ ID NO: 14 - DNA, 20 bp, synthetic construct
  source 1..20: mol_type = other DNA; note = Primer end-point PCR #1 forward
aaggaatggcaaacatttca

SEQ ID NO: 15 - DNA, 20 bp, synthetic construct
  source 1..20: mol_type = other DNA; note = Primer end-point PCR #1 reverse
ggagtcagtgggctatttca

SEQ ID NO: 16 - DNA, 20 bp, synthetic construct
  source 1..20: mol_type = other DNA; note = Primer end-point PCR #2 forward
aaggaatggcaaacatttca

SEQ ID NO: 17 - DNA, 20 bp, synthetic construct
  source 1..20: mol_type = other DNA; note = Primer end-point PCR #2 reverse
cacaccatgaccgtctcatt

SEQ ID NO: 18 - DNA, 22 bp, synthetic construct
  source 1..22: mol_type = other DNA; note = Primer end-point PCR #3 forward
tgctgaggaagatgaattagtg

SEQ ID NO: 19 - DNA, 22 bp, synthetic construct
  source 1..22: mol_type = other DNA; note = Primer end-point PCR #3 reverse
tgggatagaaatttgtgagcca

SEQ ID NO: 20 - DNA, 22 bp, synthetic construct
  source 1..22: mol_type = other DNA; note = Primer end-point PCR #4 forward
cactcgctcagctataagaaga

SEQ ID NO: 21 - DNA, 22 bp, synthetic construct
  source 1..22: mol_type = other DNA; note = Primer end-point PCR #4 reverse
tgggatagaaatttgtgagcca

SEQ ID NO: 22 - DNA, 20 bp, synthetic construct
  source 1..20: mol_type = other DNA; note = Primer end-point PCR #5 forward
cccaagtccagacacacaag

SEQ ID NO: 23 - DNA, 19 bp, synthetic construct
  source 1..19: mol_type = other DNA; note = Primer end-point PCR #5 reverse
gtggatgtggaatgtgtgc

SEQ ID NO: 24 - DNA, 19 bp, synthetic construct
  source 1..19: mol_type = other DNA; note = Primer end-point PCR #6 forward
gacaatagcaggcatgctg

SEQ ID NO: 25 - DNA, 20 bp, synthetic construct
  source 1..20: mol_type = other DNA; note = Primer end-point PCR #6 reverse
cacaccatgaccgtctcatt

SEQ ID NO: 26 - DNA, 24 bp, synthetic construct
  source 1..24: mol_type = other DNA; note = Primer porcine RT-PCR forward
acaccgataagacttcatacaact

SEQ ID NO: 27 - DNA, 24 bp, synthetic construct
  source 1..24: mol_type = other DNA; note = Primer porcine RT-PCR reverse
tgtaggttctgataccactagaga

SEQ ID NO: 28 - DNA, 24 bp, synthetic construct
  source 1..24: mol_type = other DNA; note = Primer human RT-PCR forward
ctgtgatcatcataaacagccaag

SEQ ID NO: 29 - DNA, 21 bp, synthetic construct
  source 1..21: mol_type = other DNA; note = Primer human RT-PCR reverse
aaagacagcagatgtgaggag

SEQ ID NO: 30 - DNA, 10227 bp, synthetic construct
  source 1..10227: mol_type = other DNA; plasmid = modification vector
gcttcagaaaatctatactaaaatctcaaaacatgtagatgtgaataaatgtaaactcttcaaatgcgctttatttttacatcttattccagaaatgctcagaaataaaaagaatatctctttgaaacagtatctaatgtcatacttaaatgcactcatttctttttattagattgctctggagccattaatacactaatgttcacaacaattatgctcatagagaaacggtcttaatgaaactgtatgtaagaacttgcctaagggaacagcccaaaacaagctgccccctaaactaaaatgcatgacaataaacgctagctggcaataacttcgtataatgtatgctatacgaagttatgggatccttggggtctttattgagaagtttggtgatgtttttgtgaccagaaatatgctataggaacttaactcttgtttacatcaatcagcctagggtaaaaatggttttgttatatgttgttttgcgtaaagtcatggtttccaggaacttatggaagacactgaggattactccaatacttgtagtaaatttgggagaaaaaggcaaggactaaagttactactcctaacacaaatgagacggtcatggtgtgccccatcaaaaaaggagagaaagagagacagacacatagaaacacgtgcacatacacacacacacacacacacacacacacacacacagagagagagagagagagagagagagagggagagagactatagaaacatgatgttataaataacaatagtgctgaggaagatgaattagtgtaaaattgtattttttgttctgaggataggttacatagatgattctaatccatttattatttttacattccaggctacctaaaagaagacagttatctcatatttggctgccagctttttatctttctctcgaccacttaaaacttcagacttcctgtcctgctggtatcatggagaaagtccaatacctcactcgctcagctataagaagagcctcaaccattgaaatgcctcaacaagcacgtcaaaagctacagaatctatttatcaatttctgtctcatcttaatatgtctcttgctgatctgtatcatcgtgatgcttctctgaagttctgctacaacctctagatctgcagcttgccacatcagcttaaaatctgtcatcccatgcagacaggaaaacaatattgtataacagaccacttcctgagtagaagagtttctttgtgaaaaggtcaagattaagactaaaacttattgttaccatatgtattcatctgttggatcttgtaaacatgaaaagggctttattttcaaaaattaacttcaaaataagtgtataaaatgcaactgttgatttcctcaacatggctcacaaatttctatcccaaatcttttctgaagatgaagagtttagttttaaaactgcactgccaacaagttcacttcatatataaagcattatttttactcttttgaggtgaatataatttatattacaatggaaaagcttctttaatactaagtatttttcaggtcttcaccaagtatcaaagtaataacacaaatgaagtgtcattattcaaaatagtccactgactcctcacatctgttatcttattataaagaactatttgtagtaactatcagaatctacattctaaaacagaaattgtattttttctatgccacattaacatcttttaaagttgatgagaatcaagtatggaaaagtaaggccatactcttacataataaaattccttttaagtaattttttcaaagaatcacagaattctagtacatgtaggtaaatcataaatctgttctaagacatatgatcaacagatgagaactggtggttaatatgtgacagtgagattagtcatatcactaatatactaacaacagaatctaatcttcatttaaggcactgtagtgaattatctgagctagagttacctagcttaccatactatatctttggaatcatgaaaccttaagacttcagaatgattttgcaggttgtcttccattccagcctaacatccaatgcaggcaaggaaaataaaagatttccagtgacagaaaaatatattatctcaagtattttttaaaaatatatgaattctctctccaaatattaactaattattagattatattttgaaatgaacttgttggcccatctattacatctacagctgacccttgaacatgggggttaggggagctgacaattcgtgggtccgcaaaatcttaactacctaatagcctactattgaccataaaccttactgataacataaacagtaaattaacacatattttgcgtgttatatgtattatacactatattcctacaataaagtaagctagagaaaatgttatttagaaaatcataagaaagagaaaatatatttactattcattaaatggaagtgggtcaacataaaagtcttcattctcattgtcttcacattgagtaggcagaggaggagaaagatggggaggaagagaaggcgttggtcttgcagtcttgtcttaggggtgtggggagtgggggaaagaatattcatgtataagtggacccttgcaattcaagcccttgttgttcaagggtcaactgtaataggatatagctatttttcttcctctatcaaccaaatggtaagcatctattttgtagtccactctactgagctaaattatagatccagctatgctatttataattattttcttgatgaataaattttcaatttctcctctgaccatttcagaacatcttccaataactcataaaacaactgaagtaaaattgagtgctggaaaatatattcaccaaactttggtaatttaagttgactaaagtttaaaattaagtctaaaatagtttacacctatactgcataatccaacaattttaatttcagttgaagacatgttactaatataactattattaaaagagtagaggatgtgtaattaaccatatcttctaaaacatggttactaaaagaatatgtaacatcaatattgaccttggtttcttacacaagtgttgctaactcaatagtgaaggagacactattaaattttctgaacccatgagagatactagagatggggagtggaaagtgtttggttcagggatatctgaagaacagaagggcagagatttcttaagtgacgcctcatctacaagctggaaattcctaaaaacaagtagaaagcttataaacaacaggtgatacactcacctcactggttttagtaaattaccaatacagaaagtatccctagtcttaaaaacaagtggaaaatttgaactgattagtcatattcctttgattacactgtttgttacaatatttttctcagtaaacagaaataactaatttttttgttcttcattctttgatagaaattaaaatcttattctgtgaggattacagaatactataactcaaattataaagtagaataaactctttaaataattattcttcatcataaagtgtaaagaataagatataagaaaacaatttatttttaaaatttaatatactaaatgctcaaatatgttctactatagaataagttcttatcttaatttacagggcactaaaaacaattttaaaatgcttaatgttgccttttatattttaattggttaagaatatatatttgtttaatgcaaatcagaatcactatattaaaatgaatgttcttgaaaactcagtggggctgctctataatacacttacaattgatactaacgataaaacgctctaattatagttttaattttataggggtttttttggtgactttccccaaataaaacaaaatcaagacgcccaagtccagacacacaagagggcagacgtatataagataacaaattgctactacatagcgattatttatcttttgattatttttgagacttctggcaaagtaggactataataaattttcagaaaagatctgctcttctgctaagtaacagcacacttcaaaatgtccagaaattttatgctaatattacatagaaaacttgacagatgaaaaaagtgaaacacattactaattttattcattaaaacaggtatataaaagtcctctttaactacctaatgaatgtgtcatatcctaatatagggagtgtcatagtgttcacatctttgaatatataaattatctcaaacagttaaaaaattaaagccagaacagggtacactagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttaatataacttcgtataatgtatgctatacgaagttatggatccttggggtctttattgagaagtttggtgatgtttttgtgaccagaaatatgctataggaacttaactcttgtttacatcaatcagcctagggtaaaaatggttttgttatatgttgttttgcgtaaagtcatggtttccaggaacttatggaagacactgaggattactccaatacttgtagtaaatttgggagaaaaaggcaaggactaaagttactactcctaacacaaatgagacggtcatggtgtgccccatcaaaaaaggagagaaagagagacagacacatagaaacacgtgcacatacacacacacacacacacacacacacacacacacagagagagagagagagagagagagagagggagagagactatagaaacatgatgttataaataacaatagtgctgaggaagatgaattagtgtaaaattgtattttttgttctgaggataggttacatagatgattctaatccatttattatttttacattccaggctacctaaaagaagacagttatctcatatttggctgccagctttttatctttctctcgaccacttaaaacttcagacttcctgtcctgctggtatcatggagaaagtccaatacctcactcgctcagctataagagcctcaaccattgaaatgcctcaacaagcacgtcaaaagctacagaatctatttatcaatttctgtctcatcttaatatgtctcttgctgatctgtatcatcgtgatgcttctctgaagttctgctacaacctctagatctgcagcttgccacatcagcttaaaatctgtcatcccatgcagacaggaaaacaatattgtataacagaccacttcctgagtagaagagtttctttgtgaaaaggtcaagattaagactaaaacttattgttaccatatgtattcatctgttggatcttgtaaacatgaaaagggctttattttcaaaaattaacttcaaaataagtgtataaaatgcaactgttgatttcctcaacatggctcacaaatttctatcccaaatcttttctgaagatgaagagtttagttttaaaactgcactgccaacaagttcacttcatatataaagcattatttttactcttttgaggtgaatataatttatattacaatggaaaagcttctttaatactaagtatttttcaggtcttcaccaagtatcaaagtaataacacaaatgaagtgtcattattcaaaatagtccactgactcctcacatctgttatcttattataaagaactatttgtagtaactatcagaatctacattctaaaacagaaattgtattttttctatgccacattaacatcttttaaagttgatgagaatcaagtatggaaaagtaaggccatactcttacataataaaattccttttaagtaattttttcaaagaatcacagaattctagtacatgtaggtaaatcataaatctgttctaagacatatgatcaacagatgagaactggtggttaatatgtgacagtgagattagtcatatcactaatatactaacaacagaatctaatcttcatttaaggcactgtagtgaattatctgagctagagttacctagcttaccatactatatctttggaatcatgaaaccttaagacttcagaatgattttgcaggttgtcttccattccagcctaacatccaatgcaggcaaggaaaataaaagatttccagtgacagaaaaatatattatctcaagtattttttaaaaatatatgaattctctctccaaatattaactaattattagattatattttgaaatgaacttgttggcccatctattacatctacagctgacccttgaacatgggggttaggggagctgacaattcgtgggtccgcaaaatcttaactacctaatagcctactattgaccataaaccttactgataacataaacagtaaattaacacatattttgcgtgttatatgtattatacactatattcctacaataaagtaagctagagaaaatgttatttagaaaatcataagaaagagaaaatatatttactattcattaaatggaagtgggtcaacataaaagtcttcattctcattgtcttcacattgagtaggcagaggaggagaaagatggggaggaagagaaggcgttggtcttgcagtcttgtcttaggggtgtggggagtgggggaaagaatattcatgtataagtggacccttgcaattcaagcccttgttgttcaagggtcaactgtaataggatatagctatttttcttcctctatcaaccaaatggtaagcatctattttgtagtccactctactgagctaaattatagatccagctatgctatttataattattttcttgatgaataaattttcaatttctcctctgaccatttcagaacatcttccaataactcataaaacaactgaagtaaaattgagtgctggaaaatatattcaccaaactttggtaatttaagttgactaaagtttaaaattaagtctaaaatagtttacacctatactgcataatccaacaattttaatttcagttgaagacatgttactaatataactattattaaaagagtagaggatgtgtaattaaccatatcttctaaaacatggttactaaaagaatatgtaacatcaatattgaccttggtttcttacacaagtgttgctaactcaatagtgaaggagacactattaaattttctgaacccatgagagatactagagatggggagtggaaagtgtttggttcagggatatctgaagaacagaagggcagagatttcttaagtgacgcctcatctacaagctggaaattcctaaaaacaagtagaaagcttataaacaacaggtgatacactcacctcactggttttagtaaattaccaatacagaaagtatccctagtcttaaaaacaagtggaaaatttgaactgattagtcatattcctttgattacactgtttgttacaatatttttctcagtaaacagaaataactaatttttttgttcttcattctttgatagaaattaaaatcttattctgtgaggattacagaatactataactcaaattataaagtagaataaactctttaaataattattcttcatcataaagtgtaaagaataagatataagaaaacaatttatttttaaaatttaatatactaaatgctcaaatatgttctactatagaataagttcttatcttaatttacagggcactaaaaacaattttaaaatgcttaatgttgccttttatattttaattggttaagaatatatatttgtttaatgcaaatcagaatcactatattaaaatgaatgttcttgaaaactcagtggggctgctctataatacacttacaattgatactaacgataaaacgctctaattatagttttaattttataggggtttttttggtgactttccccaaataaaacaaaatcaagacgcccaagtccagacacacaagagggcagacgtatataagataacaaattgctactacatagcgattatttatcttttgattatttttgagacttctggcaaagtaggactataataaattttcagaaaagatctgctcttctgctaagtaacagcacacttcaaaatgtccagaaattttatgctaatattacatagaaaacttgacagatgaaaaaagtgaaacacattactaattttattcattaaaacaggtatataaaagtcctctttaactacctaatgaatgtgtcatatcctaatatagggagtgtcatagtgttcacatctttgaatatataaattatctcaaacagttaaaaaattaaagccagaacagggtacactagtaaaaataaaaataagaaaactttcttttcagaggatgtagtagtatacataaactaccctcacattaaaaatagaaccttttcaaattgaaactacaatgaggtaccaactcacaccagtcagaatggccatcattagtaaatccacaaaaaataaatgctgaagagggggtggagaaaagggaaccctcctaaactgctggtgggaatgtaaattggtataactactatggaaagaagtatggaggttcctcagaaaattaaatatagaactaccctatgatccagcaatcccactcct

SEQ ID NO: 31 - DNA, 9553 bp, synthetic construct
  source 1..9553: mol_type = other DNA; plasmid = Cas9 vector
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgccaccatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgaaagggttcgatccctaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 32 - DNA, 10 bp, Homo sapiens
  source 1..10: mol_type = genomic DNA
aagaagagcc

SEQ ID NO: 33 - protein, 754 aa, Homo sapiens
ILLLAACISFVLAWFEEGEETITAFVEPFVILLILVANAIVGVWQERNAENAIEALKEYEPEMGKVYRQDRKSVQRIKAKDIVPGDIVEIAVGDKVPADIRLTSIKSTTLRVDQSILTGESVSVIKHTDPVPDPRAVNQDKKNMLFSGTNIAAGKAMGVVVATGVNTEIGKIRDEMVATEQERTPLQQKLDEFGEQLSKVISLICIAVWIINIGHFNDPVHGGSWIRGAIYYFKIAVALAVAAIPEGLPAVITTCLALGTRRMAKKNAIVRSLPSVETLGCTSVICSDKTGTLTTNQMSVCRMFILDRVEGDTCSLNEFTITGSTYAPIGEVHKDDKPVNCHQYDGLVELATICALCNDSALDYNEAKGVYEKVGEATETALTCLVEKMNVFDTELKGLSKIERANACNSVIKQLMKKEFTLEFSRDRKSMSVYCTPNKPSRTSMSKMFVKGAPEGVIDRCTHIRVGSTKVPMTSGVKQKIMSVIREWGSGSDTLRCLALATHDNPLRREEMHLEDSANFIKYETNLTFVGCVGMLDPPRIEVASSVKLCRQAGIRVIMITGDNKGTAVAICRRIGIFGQDEDVTSKAFTGREFDELNPSAQRDACLNARCFARVEPSHKSKIVEFLQSFDEITAMTGDGVNDAPALKKAEIGIAMGSGTAVAKTASEMVLADDNFSTIVAAVEEGRAIYNNMKQFIRYLISSNVGEVVCIFLTAALGFPEALIPVQLLWVNLVTDGLPATALGFNPPDLDIMN

SEQ ID NO: 34 - protein, 12 aa, Homo sapiens
CLLLICIIVMLL

SEQ ID NO: 35 - protein, 17 aa, Homo sapiens
PQQARQKLQNLFINFCL

SEQ ID NO: 36 - protein, 22 aa, Homo sapiens
KLQNLFINFCLILICLLLICII

SEQ ID NO: 37 - protein, 11 aa, Homo sapiens
PQQARQKLQNL

SEQ ID NO: 38 - protein, 7 aa, Homo sapiens
MEKVQYL

SEQ ID NO: 39 - DNA, 54 bp, synthetic construct
  source 1..54: mol_type = other DNA; note = 1st chromatogram sequence from the top in Fig. 7 (#1) - sequenced fragment of the targeting vector
tnaattttnnttatagaaattttnggatccttggggtctttattgagaagttgg

SEQ ID NO: 40 - DNA, 54 bp, synthetic construct
  source 1..54: mol_type = other DNA; note = 2nd chromatogram sequence from the top in Fig. 7 (#2) - sequenced fragment of the targeting vector
tacctcactcgctcagctataagancancctcnantgttgtgatgcctcaacna

SEQ ID NO: 41 - DNA, 54 bp, synthetic construct
  source 1..54: mol_type = other DNA; note = 3rd chromatogram sequence from the top in Fig. 7 (#3) - sequenced fragment of the targeting vector
aattaaagccagaacagggtacactagctngantgcnngncgncnnaattctac

SEQ ID NO: 42 - DNA, 55 bp, synthetic construct
  source 1..55: mol_type = other DNA; note = 4th chromatogram sequence from the top in Fig. 7 (#4) - sequenced fragment of the targeting vector
aaaaattaaagccagaacagggtacactagcttggctgcaggtcgtcgaaattct

SEQ ID NO: 43 - DNA, 55 bp, synthetic construct
  source 1..55: mol_type = other DNA; note = 5th chromatogram sequence from the top in Fig. 7 (#5) - sequenced fragment of the targeting vector
gaacccttaatataacttcgtataatgtatgctatacgaagttatggatccttgg

SEQ ID NO: 44 - DNA, 90 bp, synthetic construct
  source 1..90: mol_type = other DNA; note = 1st chromatogram sequence from the top in Fig. 10 (fragment of SEQ ID NO. 30)
ccctaaactaaaatgcatgacaataaacgctagctggcaataacttcgtataatgtatgctatacgaagttatggatccttggggtcttt

SEQ ID NO: 45 - DNA, 91 bp, synthetic construct
  source 1..91: mol_type = other DNA; note = 2nd chromatogram sequence from the top in Fig. 10 (fragment of SEQ ID NO. 30)
ccctaaactaaaatgcatgacaataaacgctagctggcaataacttcgtataatgtatgctatacgaagttatgggatccttggggtcttt

SEQ ID NO: 46 - DNA, 67 bp, synthetic construct
  source 1..67: mol_type = other DNA; note = 3rd chromatogram sequence from the top in Fig. 10 and labelled as R14 (fragment of SEQ ID NO. 30)
ccaatacctcactcgctcagctataagaagagcctcaaccattgaaatgcctcaacaagcacgtcaa

SEQ ID NO: 47 - DNA, 67 bp, synthetic construct
  source 1..67: mol_type = other DNA; note = 3rd & 4th chromatogram sequence from the top in Fig. 10 and labelled as 14del (fragment of SEQ ID NO. 30)
ccaatacctcactcgctcagctataagagcctcaaccattgaaatgcctcaacaagcacgtcaaaag

SEQ ID NO: 48 - protein, 22 aa, Homo sapiens
QYLTRSAIRRASTIEMPQQARQ

SEQ ID NO: 49 - protein, 22 aa, Homo sapiens
QYLTRSAIRASTIEMPQQARQK

SEQ ID NO: 50 - DNA, 55 bp, synthetic construct
  source 1..55: mol_type = other DNA; note = 5th chromatogram sequence from the top in Fig. 10 (fragment of SEQ ID NO. 30)
aaaattaaagccagaacagggtacactagcttggctgcaggtcgtcgaaattcta

SEQ ID NO: 51 - DNA, 76 bp, synthetic construct
  source 1..76: mol_type = other DNA; note = 6th chromatogram sequence from the top in Fig. 10 (fragment of SEQ ID NO. 30)
gcttgcggaacccttaatataacttcgtataatgtatgctatacgaagttatggatccttggggtctttattgaga

SEQ ID NO: 52 - DNA, 35 bp, synthetic construct
  source 1..35: mol_type = other DNA; note = Chromatogram fragment shown next to label "po WT" in Fig. 11
cgctctgctattagaagagcttcaaccattcaaat

SEQ ID NO: 53 - DNA, 35 bp, synthetic construct
  source 1..35: mol_type = other DNA; note = Chromatogram fragment shown next to label "po rescue" in Fig. 11
cgctcagctataagaagagcctcaaccattgaaat

SEQ ID NO: 54 - DNA, 32 bp, synthetic construct
  source 1..32: mol_type = other DNA; note = Chromatogram fragment shown next to label "po R14del" in Fig. 11
cgctcagctataagagcctcaaccattcaaat

(19) United States
(12) Patent Application Publication
(10) Pub. No.: US 2026/0305586 A1
(43) Pub. Date: Oct. 8, 2026

(54) TRANSGENIC MODEL OF HUMAN CARDIOMYOPATHY

(71) Applicants: Stichting Genetische Hartspierziekte PLN, Wieringerwerf (NL); Technische Universität München, In Vertretung Des Freistaates Bayern, München (DE)

(72) Inventors: Pieter GLIJNIS, Middenmeer (NL); Nikolai KLYMIUK, Hohenkammer (DE); Petra Runa VOCHOZKOVA, Reichertshausen (DE); Eckhard WOLF, Vierkirchen (DE)

(73) Assignees: Stichting Genetische Hartspierziekte PLN, Wieringerwerf (NL); Technische Universität München, In Vertretung Des Freistaates Bayern, München (DE)

(21) Appl. No.: 19/476,712

(22) PCT Filed: Apr. 19, 2024
(86) PCT No.: PCT/EP2024/060782

§ 371 Date: Oct. 19, 2025

(30) Foreign Application Priority Data
Apr. 20, 2023 (EP) 23169053.8

(51) Int. Cl. A01K 67/0278; C07K 14/47; C12N 15/85; G01N 33/50

(52) U.S. Cl. CPC A01K 67/0278; C07K 14/4702; C12N 15/8509; G01N 33/5088; A01K 2217/203; A01K 2227/108; A01K 2267/0375; C12N 2015/8527; C12N 2500/10

(57) ABSTRACT

A transgenic animal model is provided for studying human diseases, and in particular a transgenic ungulate is provided for studying human cardiomyopathies. For example, a transgenic ungulate is disclosed harbouring a knock-in of a transgene having at least a portion of a human PLN gene containing a mutation that results in an expression of at least a portion of a human PLN mutant protein correlated with a cardiomyopathy phenotype in human subjects. Advantageously, the transgenic ungulate as disclosed herein is a transgenic pig and/or the human PLN mutant protein is a human PLN R14del mutant protein.

### FIELD

The field of the invention relates to a transgenic animal model for studying human diseases. In particular, disclosed herein is a transgenic ungulate for studying human cardiomyopathies. For example, a transgenic ungulate is disclosed harbouring a knock-in of a transgene comprising at least a portion of a human PLN gene containing a mutation that results in an expression of at least a portion of a human PLN mutant protein correlated with a cardiomyopathy phenotype in human subjects. Advantageously, the transgenic ungulate as disclosed herein is a transgenic pig and/or the human PLN mutant protein is a human PLN R14del mutant protein.

### BACKGROUND

Cardiac disease is a leading cause of human death worldwide and genetic inherited cardiomyopathies account for a substantial proportion of these cases (Joseph et al., 2017). Cardiomyopathies are structural and functional disorders of the heart muscle and represent an important cause of sudden cardiac death. Most inherited cardiomyopathies are single gene disorders with an autosomal dominant inheritance pattern and, hence, a 50% risk of transmission to a child (Marian et al., 2017). Many of these genes encode cytoskeletal components of cardiomyocytes, which can be divided into sarcomeric and extra-sarcomeric proteins. Examples of sarcomeric proteins include α-cardiac actin, vinculin, titin, titin cap, tropomyosin, myosin heavy polypeptide 7, troponin I, troponin T, and many more. The extra-sarcomeric proteins implicated in cardiomyopathies include caveolin 1, desmin, sarcoglycan 8, lamin A/C, and, notably, phospholamban (PLN) encoded by the PLN gene.

PLN is a small regulatory protein (peptide) acting on the regulation of intracellular Ca²⁺ in cardiomyocytes. In the course of a heart-beat, Ca²⁺ is the essential messenger for contraction and relaxation of the cardiomyocyte (Chien et al., 2003). Initially, Ca²⁺ is stored in the intracellular structures of the sarcoplasmic reticulum. Depolarization of the cell causes a small influx of Ca²⁺ from extracellular storages which, in turn, causes the opening of Ryanodine Receptor (RYR) in the membrane of the sarcoplasmic reticulum, allowing a massive Ca²⁺ influx into the cytoplasm. This stimulates contraction of the cardiomyocyte myofilaments and the compaction of the cardiomyocyte. For relaxation, the ATPase sarcoplasmic/endoplasmic reticulum, Ca²⁺ transporting ion pump (e.g. being any one of the 3 major paralogs 1-3: ATP2A1 known as SERCA1, ATP2A2 known as SERCA2, and ATP2A3 known as SERCA3) transfers Ca²⁺ back into the sarcoplasmic reticulum, reconstituting the original state. PLN is an essential regulator of SERCA by temporary blocking or interfering with its pumping function, which was postulated to depend on the phosphorylation and/or oligomerization status of PLN.

A number of mutations within the translated or the regulatory regions of PLN gene have been described, each of them affecting heart function (OMIM1724059). One of these mutations involves a deletion of an entire single codon in exon 2 of the PLN gene, which results in the absence of the amino acid arginine (“Arg” or “R”) at position 14 of the PLN protein. It was proposed that this specific deletion impacts on and alters phosphorylation of PLN, which, in turn, is suspected to affect the interaction between PLN and SERCA, thus disturbing Ca²⁺ homeostasis in cardiomyocytes (Haghighi et al., 2006 and 2013). However, clear mechanistic confirmation of the above is lacking.

Like many other cardiomyopathy-related mutations, PLN R14del is a hereditary autosomal dominant mutation. It is the most frequently identified specific mutation for Dutch cardiomyopathy patients and based on a large population-based cohort study, it is classified as a recurrent and founder mutation that presumably originated between about 600-850 years ago in the North of the Netherlands from a single Frisian ancestor (van Tintelen et al., 2009). Consequently, it is sometimes referred to as the PLN “Dutch mutation”. Indeed, the R14del mutation is affecting a considerable proportion of Dutch patients with arrhythmogenic cardiomyopathy and dilated cardiomyopathy (Hof et al., 2019) and it is estimated that in the north of the Netherlands, 1 in 1500 people carry this mutation.

Once diagnosed, the patients have a poor prognosis. They are often seen to suffer from the left ventricular (LV) dysfunction and early-onset (below the age of 50) heart failure. In the case of the organ end stage failure, except heart transplantation there is no curative available for the disease. The symptoms usually manifest after puberty and include low-voltage electrocardiograms and an arrhythmogenic phenotype with a high frequency of ventricular arrhythmias, often. The cardiac magnetic resonance imaging (MRI) of the PLN R14del patients exhibits a typical pattern of scarring in the inferolateral LV wall, which is likely reflected by the negative T waves in the inferolateral ECG leads. Autopsy findings have corroborated the presence of extensive fibrosis, which may represent re-entry circuits that likely contribute to malignant arrhythmias (Hof et al., 2019).

Mouse models of the disease exist, but gave contradictory information on the phenotype (Eijgenraam et al., 2020; Raad et al., 2021). The first model, known as the “Groningen model”, is inducible and carries a floxed intact murine PLN exon 2 upstream of another murine PLN exon 2 copy harbouring the R14del-causing mutation (Eijgenraam et al., 2020). The model allows induction of a homozygous R14del genotype which results in a particularly harsh disease phenotype in mice. Humanised PLN R14del mouse models were also described (Dave et al., 2022; Rogalska at al., 2023). A model known as the “Mount Sinai model” carries the protein coding sequence of human PLN in exon 2 that is either wild-type (WT) or contains the R14del mutation. The model does not carry humanised introns or untranslated regions. Interestingly, this model appears not show a strong disease phenotype when R14del mutation is present at a homozygous state but only as a result of a heterozygous state with a genotype containing a humanized R14del-coding region and the murine WT PLN coding region as alleles (Raad et al., 2021). In sum, although genetically modified mouse models are generally preferred for many practical considerations for studying human diseases that are linked or caused by a genetic modification, in the case of human heart disease, cardiomyopathies in particular, this model appeared to have been too limited for translational research and unfortunately failed to meet the expectations.

Consequently, suitable disease models for translational research of the disease are missing, which would allow to investigate and better understand the underlying pathogenesis and research for finding life-improving and possibly life-saving solutions and medicines for the PLN R14del patients.

### SUMMARY

There is a need for the development of an animal model suitable for translational research of human cardiomyopathies. The currently existing mouse models of PLN R14del-associated cardiomyopathy have shown not to be suitable for studying the disease and for testing the future therapeutic approaches. Despite uncertainty whether transgenic animal models will be successful at all in reconstructing the diseased heart cardiomyopathy phenotype as observed in PLN mutation human carries (Doevendans et al., 2019; Vafiadaki et al., 2023), the Dutch PLN Foundation chaired by Pieter Glijnis, have set out to develop a transgenic ungulate model harbouring a human PLN mutation for gaining a better insight into the human cardiomyopathy pathomechanism and for investigating therapeutic options in a suitable pre-clinical setting for cardiomyopathy patients, in particular those carrying PLN mutations.

Several of the above-stated objectives are achieved, in a first aspect, by provision of a transgenic ungulate comprising a genome comprising at least one copy of a first transgene encoding for at least a first part of a human PLN protein.

The usability of the disclosed herein transgenic animal model for translational research on a cardiomyopathy-vulnerable human heart is herewith demonstrated below by data obtained by performing assessment of the heart tissue and function of the disclosed herein transgenic ungulate (embodied herein as a transgenic pig) model. The histological assessment showed fibrotic strains and a compacted heart muscle structure reminiscent of the human disease. Further, increased PLN staining was shown in the peri-nuclear space that is similar to the formation of PLN aggregates observed in human PLN patients (Te Rijdt et al., 2017). Importantly, split potentials in the electrophysiological mapping of the heart that form a unique signature of PLN patients, were recorded in the model animals harbouring the R14del mutation but not in the WT control animals, which correlated with abnormally increased fatty-fibrotic depositions in the interstitial tissue of the PLN R14del model animals as compared to WT animals.

In further aspects, methods of generating the disclosed herein transgenic ungulate are disclosed, as well as uses of the disclosed herein transgenic ungulate and/or its transgenic progeny and/or organs and/or cells as isolated from any one of them, in the translational research and/or in screening for therapeutic agents for use in the treatment of human cardiomyopathies.

These and other aspects of the present disclosure and advantageous embodiments thereof, together with their advantages, are explained further below.

### Definitions

As used herein, the words “comprising”, “including”, “containing” and similar wordings, are to be construed as not excluding other elements and/or steps in relation to the subject they relate to. Further, the term “comprising” should be interpreted as merely specifying the presence of the named features, elements, steps, or components referred to, but does not exclude the presence or addition of one or more other features, elements, steps or components, or groups thereof. The scope of an exemplary phrase like “a plasmid comprising gene A and gene B” should therefore not be limited to plasmids consisting only of the genes A and B. The understanding of the above exemplary phrase should be that it relates to a plasmid wherein genes A and B are present but other elements like further genes or sites can be present as well.

Further, as used in the specification and the appended claims, the articles “a”, “an”, “the”, and “said” are intended to mean that there are one or more of the elements unless the context clearly dictates otherwise. As such, the terms “a” (or “an”), “one or more”, and “at least one” can be used interchangeably herein.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Furthermore, these phrases should not be construed as indicating any kind of preference for the disclosed exemplary embodiment.

As used herein, the terms “multiple” and “plurality” in expressions like “multiple (features)” or “a plurality of (features) . . . ” are to be understood as referring to more than one of (the features). As used herein, the term “multiple” will usually refer to more than 1, such as, 2, 3, 4, 5, 6, 7, 8, 9, 10 or in the range of multiples of 10 or more, depending on the context.

As used herein, the term “and/or” indicates that one or more of the stated cases may occur, alone or in combination with at least one of the stated cases, up to with all of the stated cases.

### BRIEF DESCRIPTION OF FIGURES

For a fuller understanding, reference is made to the following detailed description taken in conjunction with reference to the accompanying Figures in which:

FIG. 1 schematically shows a transgenic animal generation procedure involving a somatic cell nuclear transfer (SCNT) technique. In the scheme, primary cells obtained from a wild-type (WT) pig are transfected with genetic constructs for genomic manipulation and engineering. Subsequently, single cell clones (SCCs) are produced by clonal selection, followed by screening for the presence of markers indicating successful genomic manipulation event. Verified SCCs are then used for injection into enucleated ova. After reconstitution, embryos are transferred to foster mothers.

FIG. 2 schematically shows a visual summary of known PLN mutations in human patients, the structure of PLN gene locus, and a multi-species sequence comparison between PLN protein orthologues in the selected species as indicated. The PLN gene contains 2 exons and is localized within a large intron of the CEP85L gene, as schematically shown in the top pane. Three alternations, indicated in the top pane, affect the regulatory elements of PLN. Three other mutations are believed to distort the protein—these mutations are indicated in the bottom pane as light grey boxes over PLN protein sequence shown in a single letter amino acid code. Among them, R14del presumably affects phosphorylation of Serine(S) at position 16 (arrow in the bottom panel). Position 27 contains a lysin (K) only in the human PLN protein, which is shown in a darker grey box in the bottom pane. Lastly, the position of the transmembrane domain is indicated (tmem). Examined species: mq-macaque, cj-marmoset, hu-human, po-pig, bo-cattle, ov-sheep, ca-dog, fe-cat, eq-horse, or -rabbit, rt-rat, mo-mouse.

FIG. 3 schematically indicates regions of interaction of PLN with SERCA1 and -2 proteins. Multi-species alignments of SERCA1, -2 and PLN (same species as in FIG. 2). The interacting amino acids in the SERCA proteins are indicated in grey boxes (upper panel) and correlated with the corresponding motifs in PLN (lower panel). The references that have identified the respective interactions are indicated for the respective motifs.

FIG. 4 shows patients-specific single nucleotide polymorphisms (SNPs) in PLN gene sequence. By correlating to the anticipated origin of the R14del mutation in a single individual in Northern Holland, deep sequencing of the PLN locus in PLN R14del patients had revealed a consistent presence of the rs12198461 and rs1051429 SNPs on the disease human allele.

FIG. 5 (A) shows a modification strategy for obtaining a transgenic pig, whereby porcine PLN exon 2 is replaced by a modification that comprise a WT variant of human PLN exon 2 and a neomycin selection cassette (neo), bordered by lox sites (triangles) as well as an R14del variant (asterisk) of human PLN exon 2. Dotted lines represent the patient-specific SNPs rs12198461 and rs1051429. The pin indicates the “G” that is placed between the lox site and the adjacent BamHI site in the WT exon 2, but not in the mutated exon 2;
(B) shows a strategy of modification of BAC CH242-318M5 by bacterial recombineering in E. coli strain SW106; (C) shows a modification strategy, first involving modification of porcine CEP85L/PLN locus in pig primary cells by CRISPR/Cas induced homologous recombination with modified CH242-318M5, followed by excision of floxed rescue-cassette+selection cassette in modified pig primary cell clones with Cre-mediated recombination

FIG. 6 shows possible optional strategic approaches for obtaining the transgenic animal as disclosed herein comprising different genetic constellations involving the human PLN transgenes. Square rectangle represents exon 2 of the PLN gene (native porcine or the human transgene) Dotted lines indicate the two SNPs rs12198461 and rs1051429 that are specific to human PLN R14del patients. The pin marks the WT-exon-specific “G” between the lox site and the BamHI site.

FIG. 7 shows a targeting vector verification strategy involving a combination of a control digest with a restriction enzyme (bottom left panel, asterisks in lane 2 and 3 indicate correct humanisation of the vector) and an end-point PCR-Sanger sequencing. The arrows in the upper panel indicate approximate position and orientation of the sequencing primers #1-#5. The electropherograms (lower right panel) indicate transitions from the humanized intron into both the porcine intron as well as into the neo selection cassette (primer #1); the abundance of the intact and the diseased human allele (primer #2); the transition from the 3′-UTR into both, the neo cassette and the downstream porcine region in the original BAC (primer #3); the transition from neo into the rescue copy (primer #4) and the transition from neo to the disease copy (primer #5; Of note: compromised Sanger sequencing pattern is common during sequencing of palindromic repeats such as those of the lox sites and is normal)

FIG. 8 shows the results of screening of SCCs. The upper panel shows a qPCR-based loss of WT-allele (LOWA) screening revealing clones with aberrant copy number ratios between the PLN locus and two reference sites (USHIC (USH) and NANOG (NG)). For better visualization, copy number ratios are inversed. A copy number ratio of 2:1 suggest one WT allele lacking (clones 14, 118, 121, marked by asterisks), an infinite copy number ratio indicates that both porcine WT alleles are lacking (clone 1). The lower panel provides a summary of the SCC screening in two cell lines. Clones that indicated an apparent lack of one WT allele were confirmed by Sanger sequencing to contain 1 remaining WT allele and had both humanized cassettes (human lox PLNWT/R14del) analysed, according the screening in FIG. 7. Finally, the number of cell clones are indicated (“total”) that met all inclusion criteria and successfully recovered from cryo-conservation for SCNT.

FIG. 9 shows a genotyping strategy for PLN transgenic pigs. Approximate positions and orientations of the end-point PCR primers are shown vis-à-vis the genetic constellations of healthy breeding animals (upper left) and diseased animals (lower left). The genotyping PCRs were designed to detect the porcine WT allele (1), the transition from the porcine sequence into the modification box (2) and WT or diseased copy of human exon 2 (3). One PCR uses a forward primer specific for the human WT, but not the R14del exon 2 (4) and two other confirm the transitions from the WT-copy to the selection cassette (neo) (5) and the transitions from neo to the diseased copy of human PLN exon 2 (5) within the rescue cassette. The right panel shows the discrimination of the founder piglets lacking the rescue cassette (#11809, #11928) from piglets with the remaining rescue cassette (#11810, #11926, #11927). Animals #12435-12440 were generated from re-cloning of the diseased founder piglets (#11809, #11928). The re-cloned #12440 was a stillborn piglet, which did not allow sufficient isolation of genomic DNA for PCR analysis.

FIG. 10 shows genetic constellations found in founder pigs. Sanger sequencing of the PCR products confirmed the modification in #11810 and the removal of the rescue cassette in #11809. PCR across the lox-site (highlighted by shaded boxes) on 5′-end (2) confirmed the abundance of a “G” (darker grey box in the second line) between lox and the BamHI site (by design part of the rescue cassette) and its lack in the mutated copy (by design part of the mutated cassette). A PCR spanning from the intronic region into the 3′-UTR (3) revealed the abundance of both, the WT and the mutated coding sequence in #11810, while piglet #11809 retained only the mutated exon. Amino acids are shown in one-letter code. Transitions from the rescue cassette into neo (5) and from neo across the downstream lox site into the mutated exon (6)) appeared only in #11810.

FIG. 11 shows qualitative expression analysis. Using RT-PCR spanning exon 1 and the coding sequence in exon 2, 3 distinct genetic constellations were discriminated. In WT pigs (upper panel), the porcine coding sequence is expressed, including the AGA triplet encoding R14 (shaded). In animals carrying the rescue cassette (middle panel), splicing was exclusive to the upstream human WT copy, including the AGA triplet encoding R14 (shaded). In animals lacking the rescue cassette (lower panel), exon 1 is spliced to the mutated human copy of exon 2, lacking the AGA. The nucleotides discriminating the porcine and human sequences of the PLN coding region are highlighted in framed boxes.

FIG. 12 shows quantitative expression analysis. Relative abundance of the humanized transcripts was determined using human- or the porcine PLN transcript-specific qPCR, suggesting that the expression level of the humanized allele (dark boxes) appears to be reduced compared to the porcine allele (light boxes) in distinct heart regions (RA: right atrium, RV: right ventricle, LV: left ventricle, LA: left atrium). Light grey-samples from an animal retaining rescue cassette (n=1 animal, 3 samples from the respective regions prepared independently); dark grey-samples from animals lacking rescue cassette (n=3), black is pig WT (n=3); y-axis-relative amount of human PLN transcripts vs porcine PLN transcripts, calculated by the delta Ct approach: 2\hat{ }(Ct (pig)-Ct (human)); in WT pig, y=approx. 0 (e.g. 0.00015+/−0.000009); which confirms the specificity of the assay.

FIG. 13 shows an analysis of putative regulatory properties. By comparing the PLN locus of 11 distinct mammalian species in an alignment of 40 kb, we observe low homology and low coverage between the species. Only small regions correlating with proposed regulatory properties (black and thickened regions) upstream and around exon 1 (highlighted framed boxes) indicate conservation within mammals. Surprisingly, the large 3′-UTR downstream of the PLN coding region has low degree of conservation between the species (last highlighted framed box) which may indicate species-specific regulatory properties.

FIG. 14 shows results of histological assessment of representative heart tissue sections obtained from a re-cloned R14del pig (upper panel) and an age-matched WT control pig (lower panel), documenting formation of fibrotic strains (arrows in Sirius red staining, left) at such early age in re-cloned R14del pig but not in the WT control. The fibrotic strains are reminiscent of the phenotype observed in human subjects carrying the R14del mutation. Furthermore, stronger HE staining suggests a more compacted tissue organization in PLN pigs compared to WT pigs.

FIG. 15 shows a histological assessment using a mouse monoclonal anti-PLN antibody (clone 2D12, Thermo Fisher) that recognises PLN from several species including human and pig, for determining PLN localisation in the human reference tissue (left panel) and a heart tissue obtained from a re-cloned PLN R14del transgenic pig (middle panel) and a WT pig (right panel), and indicating strong peri-nuclear PLN staining (arrows) only in the PLN R14del-affected transgenic pig heart tissue.

FIG. 16 shows electrophysiological malformations in the form of split potentials (arrows in panel B) that were detected only in PLN R14del transgenic pigs (panel B) but not in WT control pigs (panel A) in which the reading was as expected for a healthy heart.

FIG. 17 shows staining for fibrotic depositions by Sirius Red, which reveals that fibrotic tissue in WT animals is localised at the border zones of the myocard or around vessels (panel B), and that in the PLN R14del transgenic pigs the deposition of extracellular matrix is increased and grows into the interstitial tissue.

### DETAILED DESCRIPTION

The invention generally concerns a transgenic animal that can serve as disease model for human heart diseases, primarily involving cardiomyopathy, wherein the animal is a transgenic ungulate comprising a genome comprising at least one copy of a transgene encoding for at least a part of a human phospholamban (PLN) protein.

All technical and scientific terms as used herein have—unless specified otherwise—the same meaning as commonly understood in the field of the relevant art. Consequently, standard technical terms like genome, gene, gene copy number, diploid, haploid, chromosome, allele, mono-allelic, bi-allelic, DNA (deoxyribonucleic acid), RNA (ribonucleic acid) for example mRNA (messenger RNA), protein, transcription, translation, gene expression, gene promoter (or, in short: promoter), SNP (single nucleotide polymorphism), vector (for example a plasmid, an artificial chromosome, a viral vector etc.), PCR (polymerase chain reaction), primer, polymerase, thermocycling, transfection, infection, recombination, genotyping, genotype, phenotype, cell, somatic cell, primary cell, cell clone, muscle cell, cardiomyocyte (heart muscle cell), in vitro, in vivo, in silico, etc., which will be self-explanatory to a person skilled in the field of genetic engineering, biotechnology, molecular biology, and related domains, are to be construed as they would be understood by such skilled person. As interpretation of some specific terms may slightly vary or be subject to a nuanced perception by different individual skilled persons, several terms will be explained herein below to aid understanding of the present disclosure.

The term “ungulate” as used herein refers to a four-legged mammal with hoofs. Such defined ungulates can be divided into odd-toed ungulates (perissodactyls) and even-toed ungulates (artiodactyls). In some embodiments, the ungulate is selected from the group consisting of domestic or wild representatives of bovids, ovids, caprids, suids, equids, cervids, and camelids. Examples of such include cows or bulls, oxen, bisons, buffalos, sheep, goats, pigs, horses, ponies, donkeys, mules, deer, elks, camels, llamas, and alpaca etc. In preferred for practical reasons embodiments, the disclosed herein transgenic ungulate is a livestock ungulate. Livestock ungulates include odd-toed livestock ungulates like horses, ponies, and donkeys; and even-toed livestock ungulates like cattle (e.g. cows and oxen), goats, pigs, sheep, and some camelids (e.g. llamas, alpacas, etc.). Even-toed livestock ungulates are preferred, in particular including cattle, sheep, goats, and pigs. Even more preferred for translational science relating to human heart are medium-sized even-toed livestock ungulates because of their hearts displaying similar size and rhythmic properties to the human heart; the medium-sized even-toed livestock ungulates include pigs, sheep, and goats. In the presented herein context, the most preferred model ungulates are pigs.

As used herein, the terms “phospholamban protein”, “PLN protein”, “cardiac phospholamban protein”, and “cardiac PLN protein” are synonymous with each other and have their regular scientific meaning as referring to the protein product of the PLN gene. Human PLN protein is encoded by the PLN gene located on the chromosome 6q22.31 and containing two exons. Entrez (NCBI) Gene ID of human (H. sapiens) PLN gene is 5350 (update of 21 Mar. 2023). NCBI RefSeq protein accession no. is NP_002658 relating to the full length (FL) wild-type (WT, as used herein to be construed as non-mutated) protein product of 52 amino acids. Due to this relatively short for human protein length, PLN protein can sometimes be found in the literature to be classified as a peptide or a minipeptide. Consequently, as used herein the particular context of the PLN protein, the term “protein” is to be construed as synonymous to the terms “peptide” or “minipeptide”. The FL WT human PLN protein is entirely encoded by exon 2 of the PLN gene and is represented be the amino acid sequence shown in SEQ ID NO: 1 (cf. Table 1). A transcript of 2989 bp mRNA, which translation results in the production of the FL WT human PLN protein can be accessed e.g. under NCBI RefSeq accession number NM_002667.5, indicated to have the position of the coding side (CDS) at: 195 . . . 353 nt; exon 1 position at: 1 . . . 97nt; and exon 2 position at: 98 . . . 2989 nt.

For avoidance of any doubt, in line with the convention and as it will be evident and understood by any skilled person in the field of molecular biology and related fields, when provided herein or in the electronic listing of sequences, nucleotide sequences that represent chains of nucleotides (natural or synthetic alike), which chain may form a part of or constitute nucleic acid molecules (usually being DNA or RNA etc.) are to be assumed to be presented in the direction from 5′ to 3′ from the start of the nucleotide chain, unless indicated otherwise. Further and analogously, when provided herein or in the electronic listing of sequences, amino acid sequences that represent a chains of amino acids and which may form a part of or constitute one or more proteins, are to be assumed to be presented in the direction from N-end to C-end from the start of the amino acid chain, unless indicated otherwise.

As used herein, the term “transgene” refers to a DNA fragment represented by a sequence of nucleotides that does not naturally occur within a genome of an organism, into which genome said DNA fragment was introduced (or “knocked in”) by one or more genetic engineering techniques (usually involving so called “recombinant DNA technology”), and which DNA fragment is introduced into the genome of this organism such that it can be passed to said organism's offspring, which process can be referred to as germline (sometimes spelled as “germ line”) transmission. In line with the above definition, it can be said that the transgene introduced into an organism's genome is a DNA fragment exogenous to said organism, which can be explained as referring to a DNA fragment that is represented by a sequence of nucleotides that is not expected to be found upon sequencing of the genome into which the exogenous DNA fragment was introduced.

As used herein, the term “transgenic organism”, usually in the context of the present invention being a transgenic animal, in particular transgenic ungulate like a transgenic pig, will usually be used to refer to an organism (usually, in the present disclosure being an animal, or more specifically an ungulate, e.g. a pig, respectively and depending on the context as specified) comprising a genome comprising a transgene. However, it should be noted that the term “transgenic”, as used herein, does not have to be limited to organisms comprising a genome comprising a transgene, in other words being so called “knock-in” organisms (usually as disclosed herein, the transgenic organisms being transgenic animals) but should be construed more broadly as referring to an organism (e.g. animal, ungulate etc.) comprising a genome that has been subject to technical intervention involving manipulation by one or more genetic engineering techniques, such that said intervention has introduced a non-naturally occurring change into said genome as evident from the sequence of nucleotides representing said genome, wherein the introduced change can be passed to said organism's offspring. In addition to the above-explained knock-in organisms, another example of such defined transgenic organisms can include an organism comprising a genome wherein an endogenous to said organism DNA fragment like an entire gene or a part thereof has been deleted following the technical intervention. Such transgenic organism can be termed a “knock-out” organism with respect to the gene affected by the deletion, but as the skilled person will appreciate, a “knock-out” of a gene in a transgenic organism can also be achieved by knocking a DNA fragment into said gene and thus affecting or blocking its expression. Other examples can include transgenic organisms comprising a genome comprising a change that involved duplication of an endogenous gene or its modification within e.g. associated therewith different genomic regulatory sequences, for example in the promoter region etc. Different types of transgenic organisms comprising genomes that were subject to technical intervention involving manipulation by one or more genetic engineering techniques, including but not limited to introduction (knock in) of a transgene will be well known to the person skilled in the art and do not require further explanation.

Frequently, a transgene introduced into genome of a transgenic animal will be a DNA fragment exogenous with respect to said animal and represented by a sequence of nucleotides naturally found in a genome of another organism belonging to a different species than the species to which the transgenic animal belongs. As sometimes used herein, the organism in which genome the DNA fragment used as a transgene can naturally be found, is termed the “source organism”. For example, such source organism can be human, meaning that a transgene introduced into a transgenic animal's genome is represented by a sequence of nucleotides typically found in the human genome. For example, a transgenic animal, like the transgenic ungulate as described herein, may comprise a genome comprising a transgene encoding for a human protein or a part thereof, for example, being a DNA fragment represented by a sequence of nucleotides that is found in human genome but not found in the genome of the species to which the transgenic animal belongs.

As indicated above, the invention generally relates to a transgenic ungulate comprising a genome comprising at least one copy of a first transgene encoding for at least a first part of a human PLN protein.

In line with this, in a general concept, the disclosed herein transgenic ungulate carries a transgene in its genome that contains the genetic information to express at least a part of a PLN protein that is represented by a sequence of amino acids that is typical for the human PLN protein and is not typical to the PLN protein that is native to the species of the transgenic ungulate. For example, in an advantageous embodiment, wherein the transgenic ungulate is a transgenic pig or a transgenic goat or a transgenic sheep, the ungulate comprises a genome comprising a transgene encoding for at least a part of a human PLN protein, wherein said part is different by at least one amino acid from the corresponding orthologous PLN protein that is native to pigs, goats, or sheep, respectively.

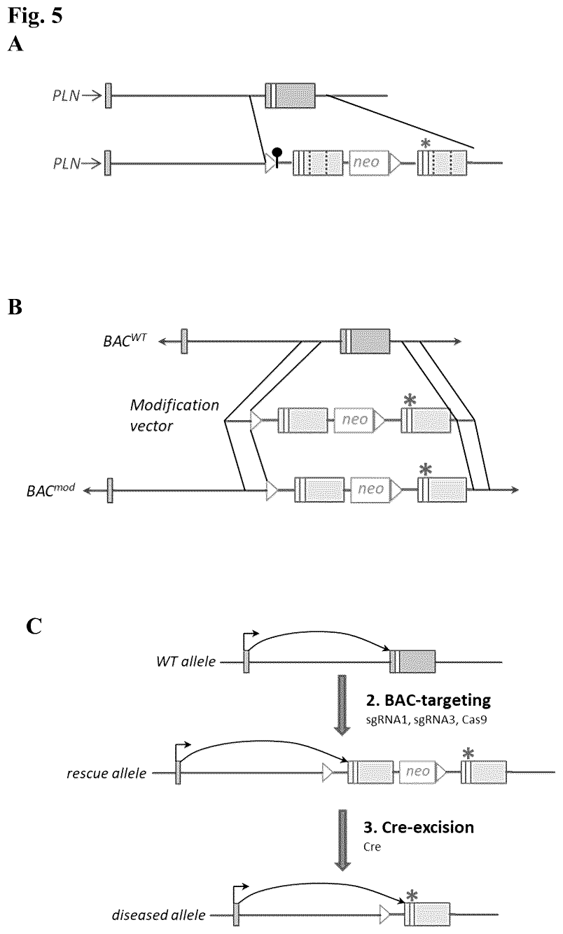

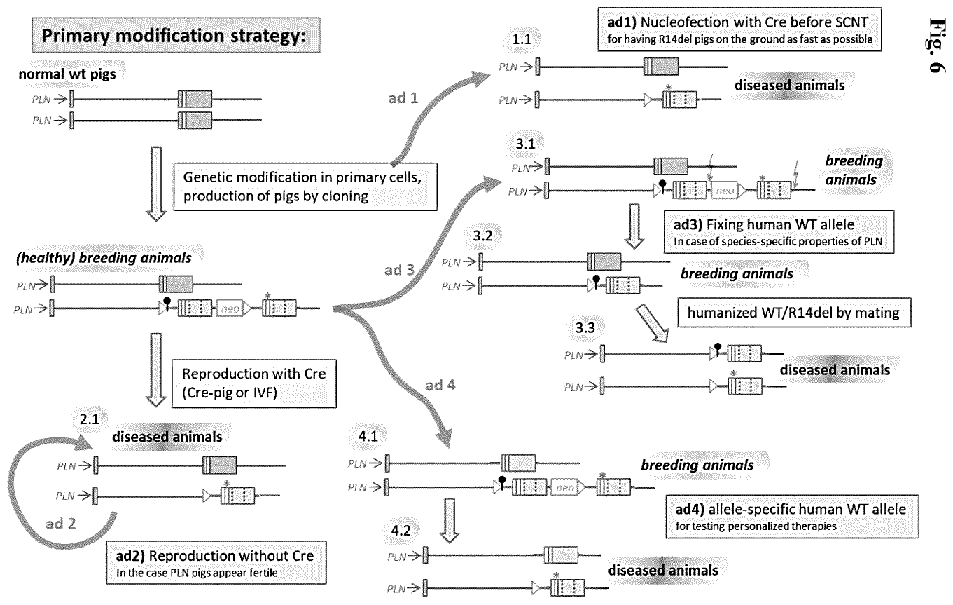

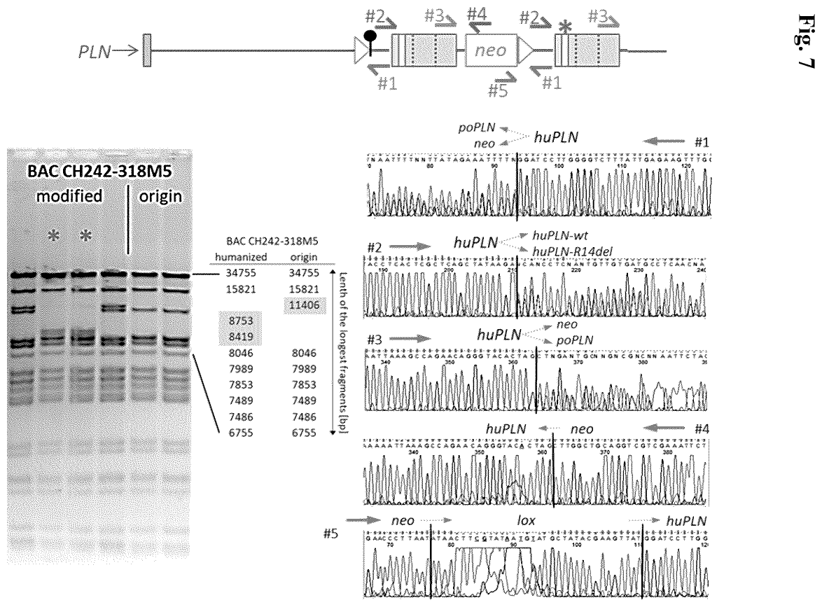

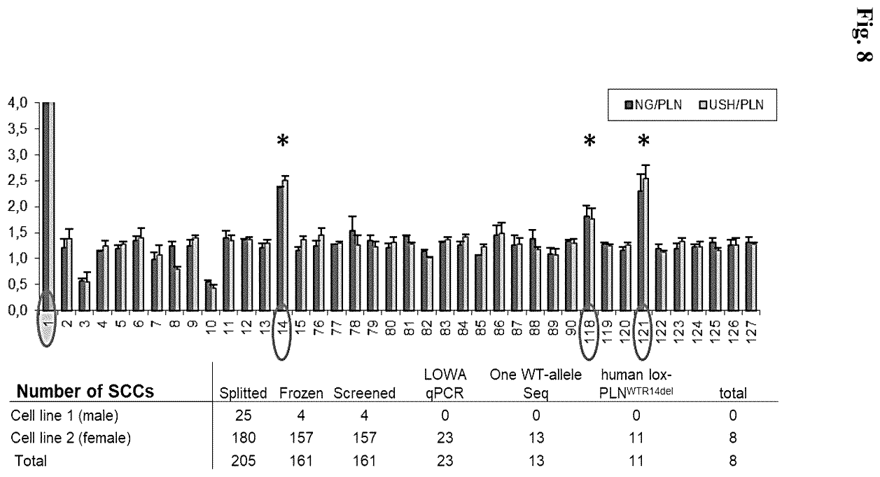

For example, the first transgene can advantageously be made of DNA represented by a sequence of nucleotides corresponding to the sequence found in a recognised human genome assembly as the reference human sequence encoding for the first part of the human PLN protein encoded by the first transgene, but it does not have to because of the degeneracy of the genetic code. For example, Ser10 of PLN protein is encoded by a TCA codon in human and in primates while the same amino acid is encoded by TCT in ungulates and carnivores. Change of the second T to A within this codon in an ungulate will introduce no difference at protein product level, but at the genomic level such differences may be exploited for genotyping purposes or for exploring human-specific therapies e.g. involving gene editing or antisense treatments. Hence, despite certain advantages, because of the presence of many synonymous codons, the DNA sequence representing the transgene does not have to perfectly match the reference human sequence to encode for a human PLN protein. For example, native ungulate PLN proteins including the porcine PLN, ovine PLN, caprine PLN, and bovine PLN proteins have aspartic acid (symbol Asp or D) at position 2 (i.e. encoded by codon 2), whereas human PLN protein harbours at this position glutamic acid (symbol Glu or E). Furthermore, human PLN appears to be unique in having a lysine (symbol Lys or K) at position 27, which is not present in other species PLN orthologues (cf. FIG. 2).

In advantageous embodiments, in particular, as explained above, for the purpose of screening for and/or testing of human-specific antisense therapeutics, the first transgene is represented by a sequence of nucleotides that is at least 80%, preferably at least 85%, more preferably at least 90%, even more preferably 95%, most preferably is identical to the corresponding reference sequence from the human PLN gene as annotated for the sequenced human genome to encode for at least the first part of a human PLN protein.

The transgene as comprised in the transgenic animal's genome, may but does not have to undergo transcription to RNA (usually, of mRNA type) encoding for a protein or a fragment thereof in the transgenic organism, or it can undergo such transcription only in an organ, tissue, or a cell type, or subpopulation of cells thereof within said transgenic organism, provided that the conditions for such transcription and subsequent expression (i.e. translation of mRNA) to a protein or fragment thereof are met (e.g. by appropriate integration into the genome of the transgenic organism, for example under an active promoter) or induced. The induction of the transgene expression can be caused by an external stimulus—e.g. caused by a human intervention providing a stimulus that induces the transgene's expression, e.g. when the stimulus is provided in the animal feed. Alternatively, the transgene expression can be triggered internally by an occurrence of a physiological condition or stage within the transgenic organism's life, possibly being differentiation of a cell population, which differentiation activates the promoter under which the transgene has been introduced into the transgenic organism's genome.

In a preferred embodiment, a transgenic ungulate is provided wherein the first transgene can be expressed in at least one cell population advantageously comprising cardiomyocytes.

In a particularly advantageous embodiment, a transgenic ungulate is provided, wherein expression of the first transgene results in production of the first part of the human PLN protein being at least a part of a human PLN R14del mutant protein comprising at least the PLN protein R14del mutation.

Usually and/or under specific circumstances, the presence of the transgene in the transgenic organism's genome will manifest as a change of the phenotype of the transgenic organism vis-a-vis the phenotype expected in a so called control organism (or a group of control organisms) from the same species as the transgenic organism, for example and usually being a non-transgenic organism or a second transgenic organism comprising a genome comprising a “WT” form of the first transgene.

In a possible embodiment, a transgenic ungulate is provided exhibiting a phenotype reminiscent of the phenotype of a human subject suffering from a cardiomyopathy, in particular a PLN mutation-associated cardiomyopathy, most preferably associated with PLN protein R14del mutation. Advantageously, a transgenic ungulate is provided exhibiting a phenotype comprising at least one of the symptoms present in the human subject suffering from a cardiomyopathy, in particular arrhythmogenic cardiomyopathy and/or dilated cardiomyopathy and/or any other or further genetically inherited PLN mutation-associated cardiomyopathies.

Frequently, a transgenic animal that is a disease model for a human single gene disease, will comprise a genome comprising a knocked-in transgene encoding for a human mutant protein or a part thereof comprising the mutation of interest. In such an instance, if said knocked-in transgene is expressed in the disease model transgenic organism and results in a specific phenotype, this phenotype can be compared and described against a control animal. The choice of a suitable control animal will vary and depend on the situation. Usually, a control animal can be a non-transgenic healthy animal from the same species expressing a WT form of a native (e.g. porcine for an animal being a pig) protein that is orthologous to the one encoded by the transgene encoding for a human mutant protein or the part thereof. Alternatively, a control animal can be a second transgenic animal comprising a genome comprising a different knocked-in transgene encoding for a human WT protein or the part thereof, which corresponds to the human mutant protein or the part thereof as expressed in the disease model transgenic animal.

As used herein, the term “mutation” has its standard meaning in the field of biological sciences and refers to any change with respect to an expected reference (usually also termed “wild-type”, “WT”) sequence of nucleotides or amino acids, which expected reference sequence would be predicted by a skilled person for a regular, usually non-diseased and/or WT organism of a given species. As used herein, the term “mutation” will usually be used to refer to change in a nucleic acid sequence, possibly visible as a deletion or an insertion (“indel”) of one or more nucleotides, or a replacement of one or more nucleotides of one type (A, T, C, G) to nucleotides of another type (e.g. a replacement of three adenines (AAA) to three thymines (TTT) or into any other three nucleotides, for example being guanine cytosine and thymine (GCT) etc.) in the mutated sequence as compared to a reference sequence in at least one position.

To avoid confusion, as used herein, in the context of products resulting from gene expression, the term “mutant protein” will be used, relating to a peptide, protein, or, when appropriately indicated, a fragment thereof and harbouring a change in an amino acid sequence as compared to a reference sequence as expected in the non-mutated state. Usually, the reference amino acid sequence will be the sequence representing a WT peptide, protein, or a fragment thereof, respectively, as it would be expected by the skilled person based on bioinformatic data e.g. in reference proteome databases.

For example, expected reference or WT sequence in a genome of an organism can be verified by the skilled person with the help of a recognised and agreed in the field genomic assembly (also termed “genome” assembly) containing bioinformatic records about genomic sequence data for said organism's species. Analogously, reference peptide or protein or fragment thereof sequences can be verified in bioinformatic protein sequence data databases. Such assemblies are publicly available and readily accessible and known to the skilled person for any of genome-, transcriptome-, and proteome-related reference and specific mutated sequences. Examples of information resources available to the skilled persons include databases of intergovernmental (e.g. European Molecular Biology Laboratory (EMBL), UniProt maintaining e.g. ExPASy database etc.), governmental (e.g. National Center for Biotechnology Information (NCBI)), or educational/academic/research (e.g. University of California Santa Cruz (UCSC)) organisations and many others.

As used herein, the term “mutant gene or a fragment thereof” is to be construed as a gene or a fragment thereof, respectively, comprising a mutation. For example “PLN gene mutant exon 2” as used herein refers to an exon 2 of the PLN gene comprising a mutation affecting one or more nucleotides as compared to a refence WT exon 2 of the PLN gene, wherein said PLN gene mutant exon 2 encodes for PLN mutant protein comprising a mutation affecting one or more amino acids as compared to a refence WT PLN protein.

As used herein, the term “PLN R14del mutant protein” is defined as a PLN protein comprising the R14del mutation, defined as a mutation involving deletion of an amino acid arginine (“Arg” or “R”, in short) at position corresponding to the position 14 of the human PLN protein. PLN R14del mutant protein, as referred to herein, can be an entirely human PLN R14del mutant protein that is represented by SEQ ID NO: 2, or it can be an entirely native to a given ungulate species PLN R14del mutant protein, or it can be a chimeric PLN R14del mutant protein, wherein the latter is to be construed as comprising at least one PLN protein part that is represented by a sequence of amino acids that the skilled person would recognise (e.g. based on sequence alignment analysis) as human and at least another part that the skilled person would recognise as non-human, e.g. as a sequence of a human PLN orthologue protein, preferably typical of an ungulate PLN protein, for example a porcine or ovine PLN protein etc., but still comprising a mutation involving deletion of an arginine corresponding to the deletion of the arginine at position 14 of the human PLN protein.

In a further advantageous embodiment, the transgenic ungulate of the disclosure is provided, wherein the first part of a human PLN protein is a full length (FL) PLN R14del mutant protein, as represented by the SEQ ID NO: 2. As human PLN R14del mutant protein is known to exert a dominant negative effect, this embodiment is advantageous for studying this effect of the full length human mutated protein to, for example, better understand processes like post-translational modifications of the FL human mutant protein and its interactions with other proteins and notably with the remaining WT PLN protein under heterozygous genetic constellation (e.g. involving mono-allelic first transgene knock-in with the second allele being WT, either native to the ungulate or WT human PLN protein if advantageously a second transgene is knocked-in and encoding for the latter). Another advantage of the expression of the FL human PLN R14del mutant protein is the fact that this allows studying active compounds that may act on the 3D conformation of the protein, thus paving the way for potential discoveries and screening of conformation-repairing therapeutics and/or epitope-specific agents like antibodies and different types thereof.

In another advantageous embodiment, the transgenic ungulate according to the disclosure is provided, wherein the first transgene comprises a first DNA segment comprising a human PLN gene mutant exon 2 that is encoding for the FL human PLN R14del mutant protein. Consequently, in an advantageous embodiment, the transgenic ungulate according to the disclosure is provided, wherein transcription of the first DNA segment results in mRNA comprising the protein coding part encoding for the FL human PLN R14del mutant protein and further comprising the 3′ UTR part (3′ untranslated region part) transcribed from the human PLN gene exon 2. This embodiment has the advantage of harbouring human genetic sequences and thus being useful not only for genotyping, but also for testing gene therapy approaches and antisense and/or transcription-silencing or modulating therapeutics.

For analogous reasons, a further embodiment of the transgenic ungulate according to the disclosure can be provided, wherein the first DNA segment further comprises a human PLN gene exon 1 or a fragment thereof. In a preferred embodiment, the first transgene would comprise both of the human exons of the human PLN gene allowing for transcription of FL mRNA encoding for the FL human PLN R14del mutant protein and containing both untranslated regions. Consequently, the human PLN R14del mutant mRNA transcribed from such first transgene would have both the 5′ and 3′ UTRs fully human. This would make it susceptible to testing of potential therapeutics that could target any one of the both UTRs or both at the same time.

In principle however, it has been hypothesised that the 3′-UTR of the PLN gene, being the longer UTR of the PLN transcripts, and also being transcribed entirely from exon 2, may likely be of higher potential interest from regulation and therapeutic intervention perspective. Namely, in human subjects carrying the R14del mutation, the 3′UTR contains two SNPs named rs12198461 and rs1051429, that are very strongly correlated with the presence of the R14del mutation. Hence, in a further advantageous embodiment, an ungulate is provided, wherein the first transgene further comprises at least one, preferably two of the SNPs selected from rs12198461 and rs1051429.

In view of the above, in an alternative embodiment, a transgenic ungulate can be provided wherein the transcript that undergoes translation to FL human PLN R14del mutant protein is a chimeric transcript comprising a part transcribed from the first transgene and a second part transcribed from the ungulate's native locus (e.g. porcine, ovine, caprine etc.) or a reminder thereof following the knock-in of the transgene and depending on how it was performed. For example, such chimeric transcript could comprise a 5′-portion corresponding the porcine exon 1 and another 3′-portion corresponding to human exon 2.

As explained above, in an embodiment that may potentially be highly advantageous from the viability and/or transgenic population propagation consideration, the transgenic ungulate is provided, wherein the expression of the first transgene is inducible. As also briefly explained above, inducibility of a transgene can be achieved by many different strategies, which will be known to the skilled person in the field of genetic engineering etc. One of such strategies makes use of constructs comprising so called cassettes.

The term “cassette” as used herein, is to be construed in accordance with the meaning this term has in the field of genetic engineering. Unless specified to the contrary, it is to be construed as an initially man-made DNA construct comprising and capable of expressing one or more transgenes, and further comprising, usually a plurality of manipulable sites, e.g. excision or recombination or transposition sites etc., allowing at least cutting out or repositioning of a DNA segment from said construct. The latter, can for example be done by an enzyme such as a restriction enzyme and/or by an enzyme-mediated excision or recombination (e.g. by an enzyme like recombinase). Typically, a cassette will comprise a transgene or a part thereof flanked by excision or transposition sites (i.e, wherein the flanked DNA is surrounded by a first excision site and a second excision site, one provided upstream and the other one provided downstream of the flanked DNA).

For example, a simple cassette can comprise a transgene and a regulatory element for regulating the expression of said transgene, wherein the regulatory element is flanked by excision sites. Such cassette can be introduced into a genome of an animal. At a desired moment, excision of the regulatory element may be induced, thus affecting the expression of the transgene, by e.g. either activating it (excision of an expression-blocking element), or deactivating it (excision of an expression-driving element), or otherwise modulating it (e.g. by creation of an expression regulating element responsive to a specific transcription factor etc.).

Alternatively, another transgene can also be a regulatory element for a different transgene present in a cassette. In such example, excision of the first (upstream) transgene can be used to activate the expression of the second transgene e.g. positioned downstream of the first transgene, wherein the terms upstream and downstream are defined with respect to the direction from which the transcription is initiated. This can for example be a natural promoter sequence under which such cassette is integrated in to the organism's genome, or it can be an exogenously cloned promoter sequences already included in the cassette prior to its integration in the genome.

A typical example of a cassette is a so called “selection cassette”, which term, as used herein refers to a cassette that enables the selection of genetically modified single cell clones into which genome the cassette has successfully been introduced under the selection conditions. For example, the selection can encode for an antibiotic resistance allowing selection on an antibiotic-containing medium. Thanks to the cassette's excision, once the resistance-conferring transgene is not needed, it can be removed.

As used herein “rescue cassette” refers to a cassette that includes a rescue transgene that is a transgene provided to at least temporarily counteract detrimental or in general undesirable effects e.g. on the health of the transgenic animal comprising the rescue cassette, which effects are expected to occur if the rescue transgene is removed from the rescue cassette thanks to its excision functionality. In a frequent situation, a rescue transgene can be any DNA fragment that at least temporarily blocks an expression of another gene or transgene e.g. encoding for a mutant protein, which expression is expected to cause detrimental effects like a challenging phenotype (e.g. lethal, possibly embryonic lethal, or infertility that prevents or interferes with transgenic population propagation). Thanks to the excision functionality provided by the cassette, the rescue transgene can be permanently or temporarily removed, thus allowing to study the mutant protein at a desired stage. For example, this stage can be initiated once the transgenic animal reaches puberty and perhaps already had a chance to have offspring etc. In that sense, rescue cassettes can be described as providing the “rescue” from the undesired phenotype. Frequently, rescues cassettes may comprise a rescue transgene encoding for a WT protein, and which WT transgene is expressed for as longs as it is not e.g. excised to trigger expression of e.g. the mutant transgene.

A construct functioning in an opposite manner to a rescue cassette can be termed a “disease-causing cassette”, or, in short, a “disease cassette” which, as used herein, shall be construed as relating to a cassette comprising a transgene encoding for a disease-causing protein.

As the human PLN R14del mutant protein adversely affects the function of the WT PLN gene product, the mutant allele with the deletion of the codon 14 is said to exert a dominant-negative effect, typical for most of the single gene mutations linked to cardiomyopathies. In view of the dominant-negative effect, and independently from the rescue strategy considerations, in an advantageous embodiment, a transgenic ungulate is provided comprising a genome comprising two different transgenes each one comprising a different allele and encoding for a different human PLN protein, the first allele associated with the human PLN R14del mutant protein and the second allele associated with the human WT PLN protein. The advantage of such embodiment is that in case both of these transgenes are expressed, it will allow studying the dominant-negative effect and pathological consequences thereof as observed in the human patients. Furthermore, and regardless of the expression status, in a related embodiment of the transgenic ungulate comprising a genome comprising such first and such second transgene, the related embodiment can be used as valuable breeding transgenic animal for generating transgenic ungulate progeny displaying different genetic constellations (e.g. knocked out for the native ungulate PLN gene expression) for investigating different pathological processes underlying human cardiomyopathy. Consequently, in an advantageous further embodiment, a transgenic ungulate of the disclosure is provided, wherein the genome further comprises at least one copy of a second transgene encoding for at least a part of a WT human PLN protein.

In line with the above, in a further embodiment, a transgenic ungulate is provided, wherein the expression of the second transgene results in production of the part of a WT human PLN protein. Such part of the WT human PLN protein can be defined as a part of a PLN protein which differs from the corresponding part of a WT native to the given ungulate (e.g. equine, bovine, hircine (caprine), ovine, or porcine etc.) PLN protein by at least one amino acid that is typical for a WT human PLN protein (e.g. K at position 27 or E at position 2). Preferably, such transgenic ungulate is provided wherein the part of the WT human PLN protein is a full length (FL) WT human PLN protein. As explained above, such a transgenic ungulate expressing both the FL WT human PLN protein in addition to expressing the PLN R14del mutant protein can be very valuable for translational research, in particular when provided as “fully humanised” with respect to the PLN protein embodiment, wherein the expression of its native (e.g. equine, bovine, hircine (caprine), ovine, or porcine etc.) ungulate PLN protein has been disabled, possibly at least in the heart, thus only allowing human PLN protein variants and effects linked thereto to be researched.

In a further advantageous embodiment, the transgenic ungulate is provided wherein the second transgene comprises a second DNA segment comprising a WT human PLN gene exon 2, optionally wherein the second segment further comprises a WT human PLN gene exon 1. Analogously, in further particular embodiment, such transgenic ungulate can provided as “fully humanised” on genetic level transgenic ungulate wherein also the first DNA segment comprised in the first transgene further also comprises human PLN gene mutant exon 2, without or, advantageously also with human PLN gene exon 1.

In another particular and advantageous embodiment, the transgenic ungulate can be provided wherein the second transgene is comprised as part of a rescue cassette that is preferably positioned upstream of the first transgene, more preferably wherein the rescue cassette is adapted to be removable by Cre recombinase-mediated DNA recombination, more preferably wherein the removable rescue cassette comprises lox (e.g. loxP, loxN, and lox2272 etc.) or similar excision sites flanking (aka “floxing”) the transgene 2.

Cre recombinase-mediated DNA recombination is an established method for site-specific genetic manipulation via recombination of DNA using an enzyme tyrosine recombinase based on a Cre-recombinase from P1 bacteriophage, which method is known in the field of genetic engineering and e.g. allows conditional control of gene expression in e.g. animal models. Cre recombinase is frequently used in combination with lox or types thereof (e.g. loxP, lox2272, etc.) recombination sites wherein the Cre-mediated recombination takes place, leading to genetic manipulation events such as deletion, translocation, or inversion, depending on localisation and/or orientation and/or number of lox or lox-like recombination sites.

Cre-recombinase and similar (e.g. the S. cerevisiae-derived Flp-FRT) systems allow flexible design of genetic manipulation events in transgenic animals. For example a different transgenic ungulate line can be provided, which is expressing Cre recombinase under e.g. a tissue-specific promoter, e.g. in the heart and e.g. only as of certain age, e.g. adulthood. Upon crossing such heart-specifically Cre-expressing transgenic ungulate with an embodiment of an ungulate as disclosed herein, which embodiment comprises a genome comprising a rescue cassette blocking expression of a human PLN protein R14del mutation through a floxed (e.g. flanked by lox sites) expression-inhibiting DNA element, a transgenic ungulate progeny will be obtained that will express the human PLN protein R14del mutation only in the heart of such transgenic progeny ungulate and only in adulthood. Such and other configuration of inducible expression systems will be easy to design in view of the presented herein teachings and common general knowledge of genetic manipulation of animals, and hence will be possible to combine with the presented herein transgenic ungulate embodiment examples, in particular the ones comprising transgenes provided as part of cassettes.

In a possible embodiment, the first transgene can also be provided comprised as part of a cassette (which, as used herein, can be termed “disease cassette” for being associated with a disease-causing allele) and flanked by excision and/or recombination sites, more preferably being different sites from the ones present as part of the rescue cassette comprising the second and WT-allele associated transgene. Such an embodiment can be advantageous for generating humanised WT PLN transgenic ungulates that are useful not only for researching WT PLN protein in an animal model but also for performing crosses with other embodiments of the transgenic ungulate as disclosed herein to obtain different, possibly advantageous, transgenic ungulate diseases models having different genetic constellations with respect of different human PLN-related transgenes.

In a further embodiment that is advantageous for genotyping, the transgenic ungulate is provided wherein the removable rescue cassette further comprises a site (in its DNA, e.g. like a SNP) that is represented by a unique sequence allowing discrimination by genotyping between the removable rescue cassette being comprised within the genome or removed therefrom. Further features that will be standard for the skilled person, such as additional markers, selection cassette, restriction digestion sites etc. can further be included in the transgenic ungulate embodiments as disclosed herein. Such features, when assessed as advantageous under a given setting or genetic constellation by the skilled person, can further be included in accordance with the art. An example of such an optional feature can be e.g. an embodiment wherein the removable rescue cassette further comprises a selection cassette, etc.

As already briefly explained above, in an embodiment, the transgenic ungulate is advantageously provided, wherein the first transgene, and preferably also the second transgene, are knocked-in into the locus of native PLN gene. More preferably, a specific embodiment can be provided wherein the native PLN gene exon 2 is replaced as a result of said knock-in by at least the first transgene, most preferably wherein the transgenic ungulate comprises a mono-allelic replacement of the native PLN gene with at least the first transgene, and optionally also a second mono-allelic replacement of the other PLN gene with the second transgene, thus arriving at the heterozygous constellation as seen in PLN human patients.

In a further but related embodiment, the transgenic ungulate is provided, comprising a first mono-allelic knock-in with the first transgene, and preferably further comprising a second mono-allelic knock-in with the second transgene, more preferably wherein the transgenic ungulate comprises both of the native (as used herein, native with respect to the given ungulate species, like porcine, ovine, bovine, caprine etc.) PLN gene alleles knocked-out.

In an alternative embodiment, the transgenic ungulate is provided comprising both of the native PLN gene alleles knocked-out and/or replaced as a result of a knock-in with the first transgene. In this embodiment, the transgenic ungulate is homozygous for the first transgene, which could have some use for possibly accelerating the pathological process for research reasons and/or providing homozygous research material for certain strategies of screening for the mutation-specific compounds.

In further advantageous in view of maintenance embodiments the transgenic ungulate as disclosed herein can be a transgenic livestock ungulate. Preferably, from the perspective of translational organ research, the transgenic livestock ungulate will preferably be a medium-size ungulate, preferably a medium-size livestock ungulate selected from a pig, a sheep, or a goat, and, hence, having a heart similar in size and many functional and anatomical properties to the human heart, and thus suitable for modelling pathological processes of underlying human cardiomyopathies, as well as researching treatments thereof.

Advantageously, the disclosed herein transgenic ungulate is a transgenic even-toed livestock ungulate, more preferably being selected from any one of a transgenic pig, transgenic sheep, transgenic goat, and transgenic cattle, more preferably being a medium-size even-toed livestock ungulate selected from any one of transgenic pig, transgenic sheep, and transgenic goat, most preferably being a transgenic pig.

In an further and related embodiment, a transgenic ungulate of the disclosure is provided obtained or obtainable by a method comprising the steps of:
(a) genetic modification of primary ungulate cells (e.g. primary pig, or primary sheep, or primary goat etc. cells);
(b) verification of single cell clones genetically modified in (a) for the presence of a signal that can be correlated with the at least first transgene and/or the second transgene;
(c) use of the single cell clones selected following the verification in (b) for somatic cell nuclear transfer.

As it will be appreciated by the skilled person in the field of genetic engineering, the steps of the above described method are rather classical and can be performed using any further genetic engineering techniques or modification thereof as known in the art. For example, the step (a) will possibly comprise a somatic cell nuclear transfer, which term as used herein has its regular meaning in the field, and is referring to a process, frequently used in genetic engineering and/or cloning of animals and involving a transplantation of somatic cell nucleus into usually an enucleated oocyte Another possibility would be complementation of embryos with genetically modified pluripotent stem cells to generate germ-line chimeras transmitting the transgene to the next generation. An example of how an embodiment of the disclosed herein transgenic ungulate, being a transgenic pig model, is provided in detail in the Examples section, but other techniques and approaches can also be used.

In a specific embodiment, the transgenic ungulate is provided as obtained or obtainable following the above-described general method, wherein the method further comprises the step of inducing the expression of the first transgene by removing the removable rescue cassette comprising the second transgene either:
in vitro, preferably in primary ungulate cells, more preferably by a vector transfection or viral vector infection (e.g. transfection with Cre) or e.g. by micro-injection of Cre protein or Cre-encoding mRNA e.g. into a zygote carrying the described herein rescue cassette; or
in vivo by performing a cross with a second transgenic ungulate (of the same ungulate species, e.g. a second transgenic pig) comprising a second genome comprising a third transgene encoding for means adapted to remove the removable rescue cassette following the third transgene's expression. (e.g. Cre pig).

In a further aspect, the disclosure also provides a method of generating the transgenic ungulate according to any one of the described herein embodiments, the method comprising the steps of:
(a) genetic modification of primary ungulate cells (e.g. primary pig, or primary sheep, or primary goat etc. cells);
(b) verification of single cell clones genetically modified in (a) for the presence of a signal that can be correlated with the at least first transgene and/or the second transgene;
(c) use of the single cell clones selected following the verification in (b) for somatic cell nuclear transfer.

In a specific particular embodiment of the above aspect, a method is provided, further comprising a step of inducing the expression of the first transgene by removing the removable rescue cassette comprising the second transgene either
in vitro, preferably in pig primary cells, more preferably by a vector transfection or viral vector infection (e.g. transfection with Cre) or e.g. by micro-injection of Cre protein or Cre-encoding mRNA e.g. into a zygote carrying the described herein rescue cassette; or
in vivo by performing a cross with a second transgenic pig comprising a second pig genome comprising a third transgene encoding for means adapted to remove the removable rescue cassette following the third transgene's expression. (e.g. a Cre-expressing transgenic pig).

In a specific aspect of the present disclosure, a progeny of the transgenic ungulate as disclosed herein is provided, advantageously being a progeny of the transgenic ungulate as obtained or obtainable following any possible embodiment of the above disclosed method generating the transgenic ungulate, or being a direct (sometimes termed in the art as the first generation or F1) or more distant progeny of said progeny (sometimes termed in the art as the second or further generation or e.g. F2, F3, F4 etc.), wherein the genome of the progeny comprises at least the first transgene or the second transgene.

In line with the above, the present disclosure also provides a progeny of the transgenic ungulate as described herein, wherein the progeny is also transgenic, which means that the transgenic progeny is also a transgenic ungulate comprising a genome comprising at least the first transgene an/or the second transgene. Possibly, the disclosed herein progeny of the transgenic ungulate of the disclosure, which progeny is transgenic is to be understood as comprising at least of the first transgene or of the second transgene or of both the first and the second transgene as disclosed herein. For the disclosed herein progeny that is transgenic, to be construed as forming part of the present disclosure, the consideration is whether any one of the first and/or the second transgene is comprised in said progeny's genome in a germline transmissible manner (i.e. that can be passed to a further generation progeny, such that said further generation progeny is also transgenic with regard to the presence of the first transgene and/or the second transgene), regardless of said transgene's copy number in said progeny's genome (e.g. single copy, duplicated, triplicated etc.), the transgene's transcriptional activity and/or expression status (e.g. undergoing expression in at least one organ, tissue-type, cell type etc., which expression can be transient or non-transient and/or that is or has been subject to activation in an inducible manner etc., alternatively wherein any one of the first transgene or second transgene appears to be latent, which is to be construed that no transcript or expression product associated with said transgene can be considered detected within a detection limit as set for the detection methods applied to detect said transcript or expression product), or its genetic constellation (mono-allelic, bi-allelic, positioned in cis, positioned in trans etc.).

In a next aspect, the present disclosure also provides an organ (understood as the organ comprising the transgene) isolated from the transgenic ungulate according to any one of disclosed herein embodiments, and/or the progeny that is transgenic as explained above, preferably wherein the organ is a heart.

In a yet another aspect, the present disclosure further provides a cell (understood as the cell comprising the transgene) isolated from the transgenic ungulate according to any one of disclosed herein embodiments, and/or the progeny that is transgenic as explained above, or from the isolated organ as defined above, preferably wherein the cell is a cardiomyocyte.

In a further aspect, a use of the transgenic ungulate or its transgenic progeny according to any one of the disclosed herein embodiments, or of the isolated organ as defined above, or of the isolated cell as defined above, is provided for translational research and/or as a human heart disease model system, preferably in translational research for studying human heart disease mechanism and/or progression, preferably wherein the human heart disease is a cardiomyopathy (i.e. heart myopathy), in particular arrhythmogenic cardiomyopathy and/or dilated cardiomyopathy and/or any other or further genetically inherited phospholamban-associated cardiomyopathy, most preferably associated with PLN protein R14del mutation.

Preferably, the use is provided wherein the translational research involves studying and/or correcting arrhythmogenic properties of the heart and/or heart pathologies associated with PLN protein R14del mutation.

Advantageously, the use is provided comprising administration of a biologically active agent, preferably being a nucleic acid or a small molecular compound, more preferably wherein the biologically active agent is a nucleic acid for gene therapy and/or can be a nucleic acid being an oligonucleotide therapeutic (that can chemically be an oligonucleotide based on DNA or RNA, or can be a modified, and/or synthetic oligomer comprising any one or more of the non-natural oligonucleotide-like molecule chemistries, like the ones seen in e.g. morpholino oligomers, peptide nucleic acids, locked nucleic acids, etc.). Alternatively, the biologically active agent can be small molecular compound, for example being a Ca²⁺-scavenger.

The term “biologically active agents” has its standard meaning and includes chemical substances or their formulations that are not biologically inert, meaning which can affect biochemical processes and/or interactions that are happening within a living organism, usually within at least some of said organism's cells. Usually, as used herein, the term “biologically active agents” will relate to chemical substances or their formulations that are intended for use in curative or research purposes, including but not limited to treatment, prevention, diagnosis etc. of a disease or a physiological state affecting a human or an animal.

In yet another aspect, the present disclosure also provides a method of screening biologically active agents for identification of a therapeutic agent for use in the treatment of a human heart disease preferably being a heart myopathy, in particular arrhythmogenic cardiomyopathy and/or dilated cardiomyopathy and/or any other or further genetically inherited phospholamban cardiomyopathy, most preferably associated with PLN protein R14del mutation, the method comprising at least the steps of:
administration of at least one biologically active agent at least once either in vivo to the transgenic ungulate as disclosed herein, or its progeny that is transgenic as disclosed herein, or in vitro into the isolated organ of the disclosure or the isolated cell according to the disclosure, usually followed by
observation of effects caused by said administration.

In a particular embodiment of the disclosed herein method of screening biologically active agents, the method is provided wherein observation of an improvement of at least one property associated with the function or structure of the heart, heart tissue, and/or cardiomyocytes, wherein the property can e.g. be selected from any one of: arrhythmogenic property of the heart or of cultured cardiomyocytes, Ca²⁺ influx, size and/or anatomic features of the heart etc., indicates identification of a therapeutic agent for use in the treatment of a human heart disease preferably being a heart myopathy, in particular arrhythmogenic cardiomyopathy and/or dilated cardiomyopathy and/or any other or further genetically inherited phospholamban cardiomyopathy, most preferably associated with PLN protein R14del mutation

For example, the disclosed herein screening method may comprise a step of administering a candidate therapeutic agent to the transgenic ungulate of the disclosure, followed by at least one step of monitoring the transgenic ungulate for at least one or more specific symptom of a cardiomyopathy, preferably being an PLN-associated cardiomyopathy, wherein the detection of an improvement in at least one, preferably more than one, advantageously all of said symptoms, indicates an identification of a compound that can be used for treatment of a human heart disease.

And further, the present disclosure also provides a therapeutic agent for use in the treatment of a human heart disease preferably being a heart myopathy, in particular arrhythmogenic cardiomyopathy and/or dilated cardiomyopathy and/or any other or further genetically inherited phospholamban cardiomyopathy, most preferably associated with PLN protein R14del mutation, wherein the therapeutic agent is identified by performing the disclosed herein method of screening biologically active agents according to any one of the disclosed herein embodiments.

### EXAMPLE

The inventors aimed at generating a transgenic animal model for studying and developing treatments for human cardiomyopathies. To do so, they have set out to generate a PLN gene mutation-linked cardiomyopathy model that facilitates the broadest possible usage for pre-clinical research. As hearts of medium-sized livestock ungulates, in particular pig hearts, are relatively similar in size, structure, and function to the human heart, a pig model was selected, despite the initial concern whether a human-specific phenotype associated with a PLN mutant protein could be mimicked by an ungulate transgenic animal model. For consistency, the cardiomyopathy transgenic pig model, which generation is presented hereinbelow, will further be referred to as the PLN pig or the transgenic PLN pig.

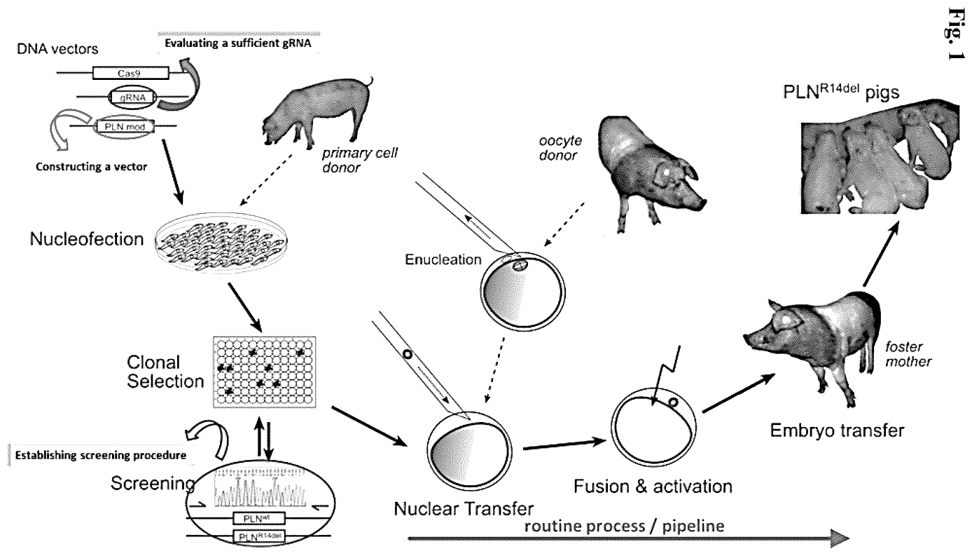

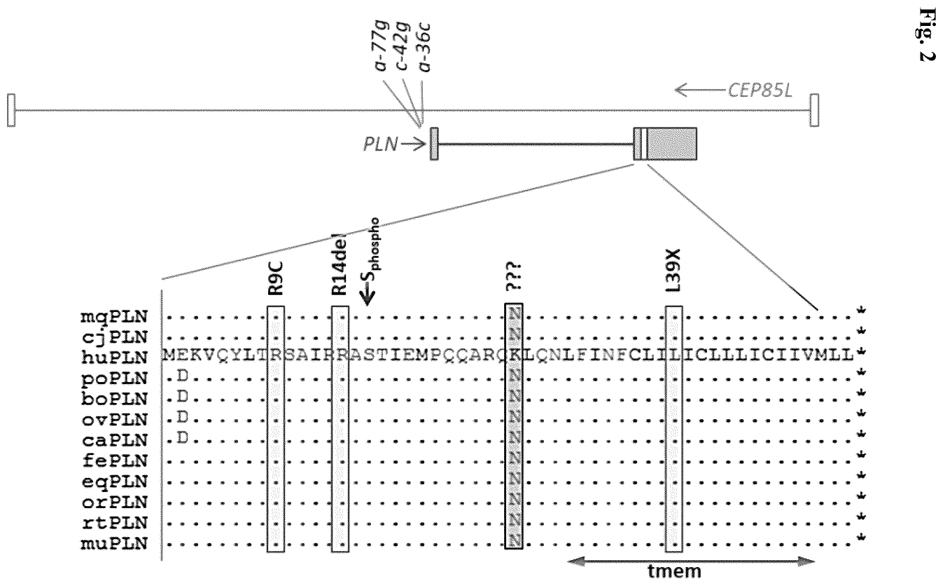

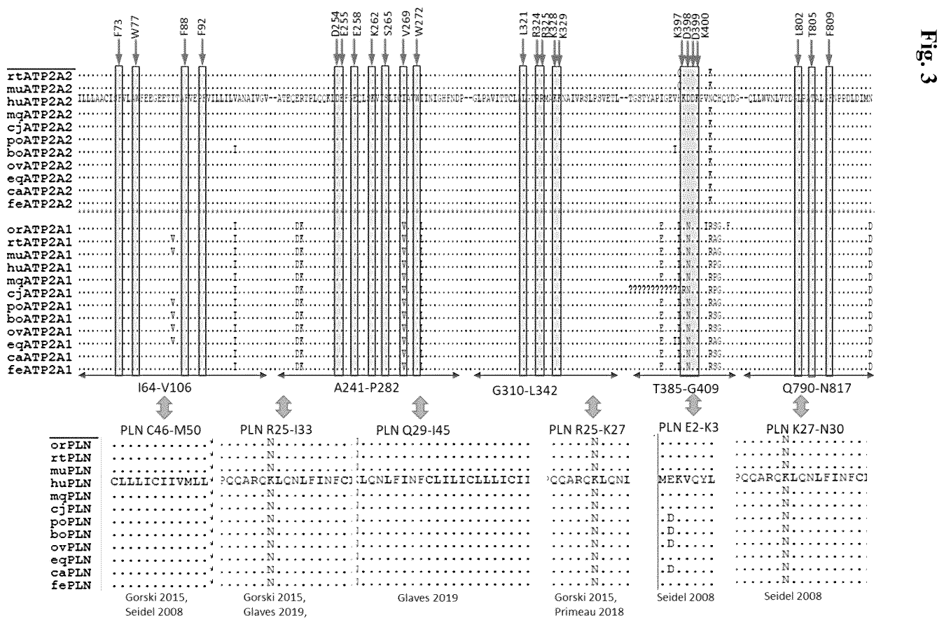

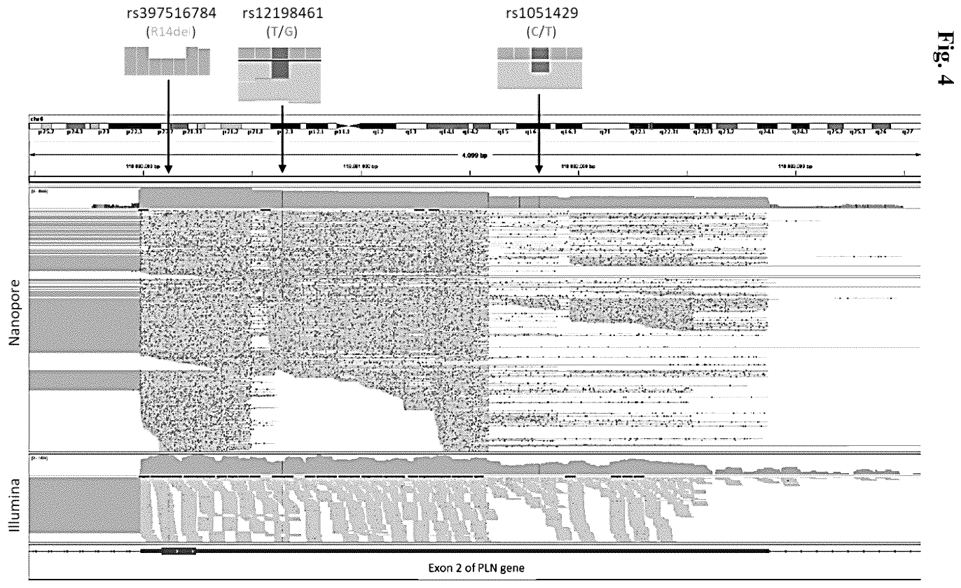

To ensure maximal viability of the animals for breeding purposes and to maximise the chances of maintaining the transgenic population once obtained, given the initial uncertainties of the final phenotype, use of a removable rescue cassette was envisaged. The transgenic animal generation procedure first involved a genetic modification of pig primary cells, followed by verification of single cell clones for the presence of the desired modification, and then, use of the verified cells in somatic cell nuclear transfer (Kurome et al., 2015; Vochozkova et al., 2019) as shown in FIG. 1.

### Design of the Model

In the first step, the PLN protein amino acid sequence conservation level was analysed and compared across selected mammalian species. The comparison is shown in FIG. 2 and shows that the PLN protein, despite being a short peptide, is relatively diverse across the examined species. The human PLN protein has a unique lysine (Lys, K) residue at position 27, while PLN protein of every other species included in the analysis has an asparagine (Asn, N) at the corresponding position. Mutations found in PLN patients, however, affect amino acids that appear conserved among the examined species.

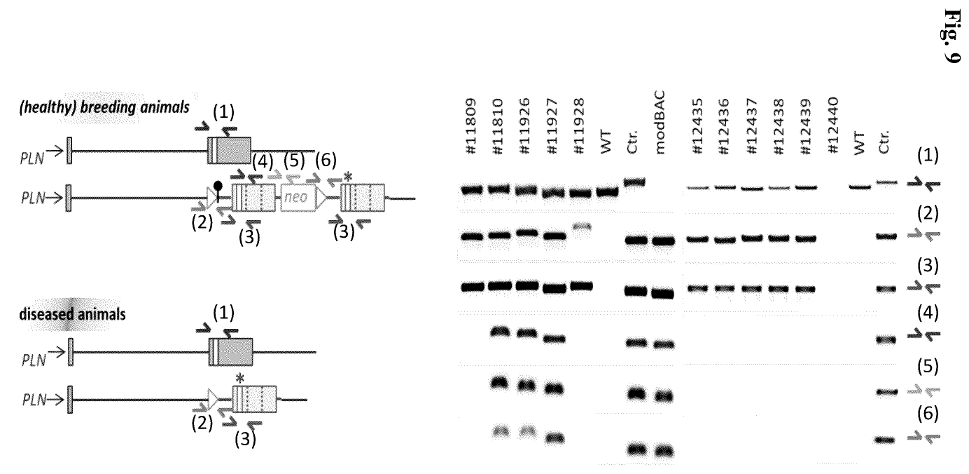

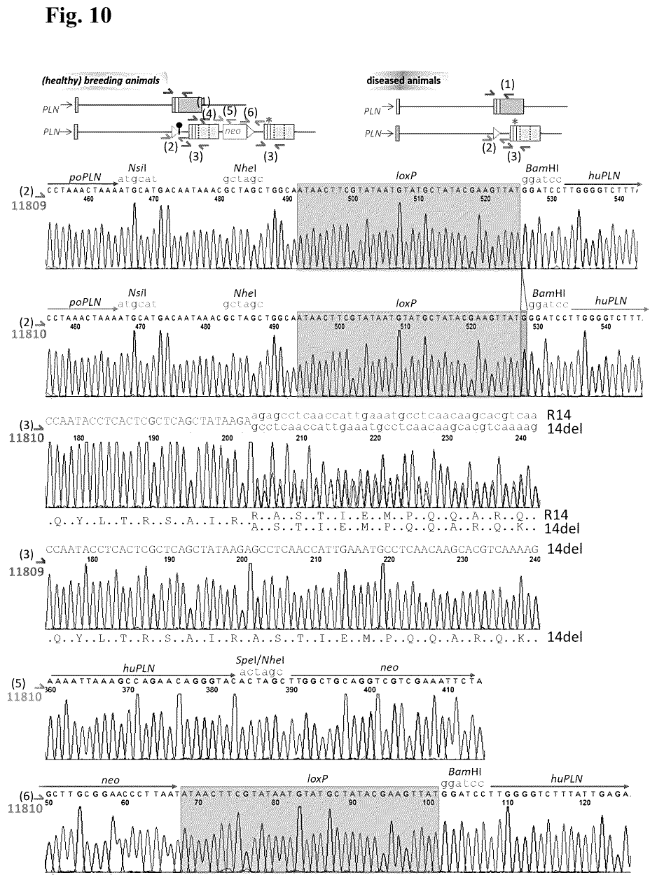

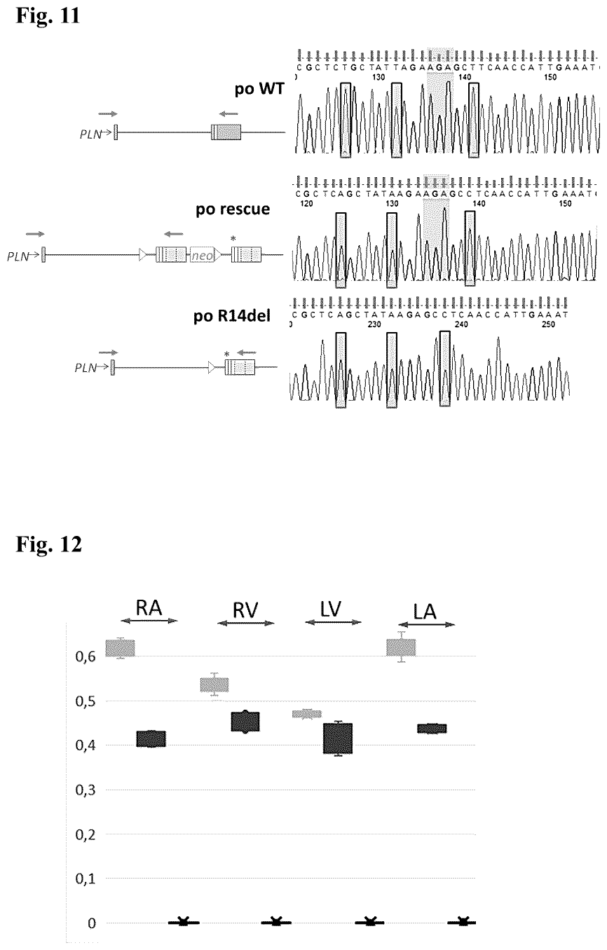

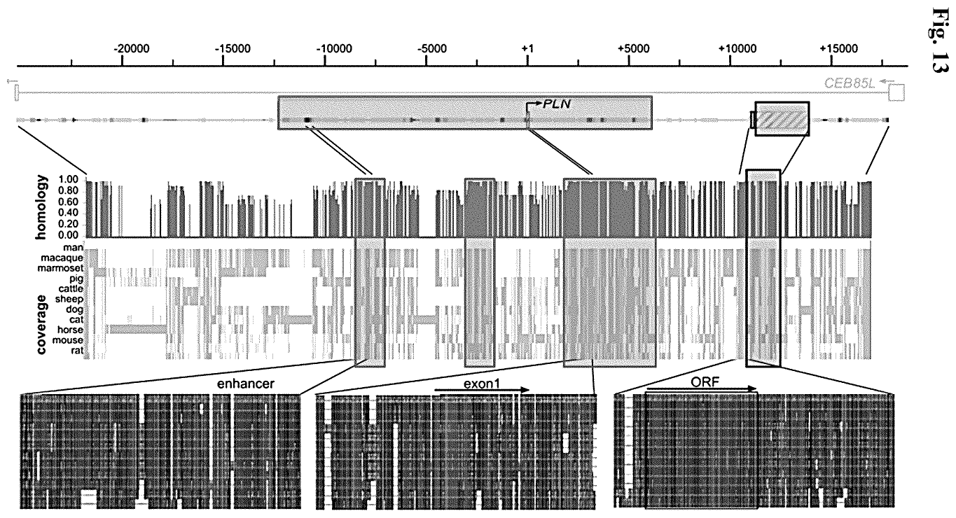

To estimate potential effects of humanization of the PLN amino acid sequence in the future transgenic pig model, localisation of PLN's known binding partners, SERCA1A1 and SERCA1A2 was investigated. In contrast to PLN, the binding sites in both of the SERCA proteins appear absolutely conserved among the species, as shown in FIG. 3, suggesting that a humanized PLN protein will act on the porcine SERCA in a similar manner as the native porcine PLN protein.

Then, the genetic modification strategy was assessed vis-a-vis potential treatment options. Beyond pharmacological approaches, the dominant negative nature of the mutated PLN advises selective blocking of transcripts generated from the mutated allele, either by specific mRNA downregulation or specific manipulation of the gene. In particular, the genetic therapy by gene manipulation appears promising as cardiomyocytes represent a cell population with limited renewal potential. Therefore, they mostly remain throughout life, making the cure of the disease at the genomic level highly attractive. Due to the fact that the genetic code is degenerated (i.e., 20 amino acids are encoded by 61 triple-nucleotide codons), the diversity at the genomic level is normally higher than at the protein level. With this in mind, a decision was made to complete humanization of the PLN coding region. However, it was not sure that the human PLN R14del mutant protein product resulting from this region's humanisation will undergo in the pig cells the same phosphorylation, protein-protein interactions, and homo-oligomerization with the WT protein as the endogenous human PLN R14del mutant protein does in the human cells.

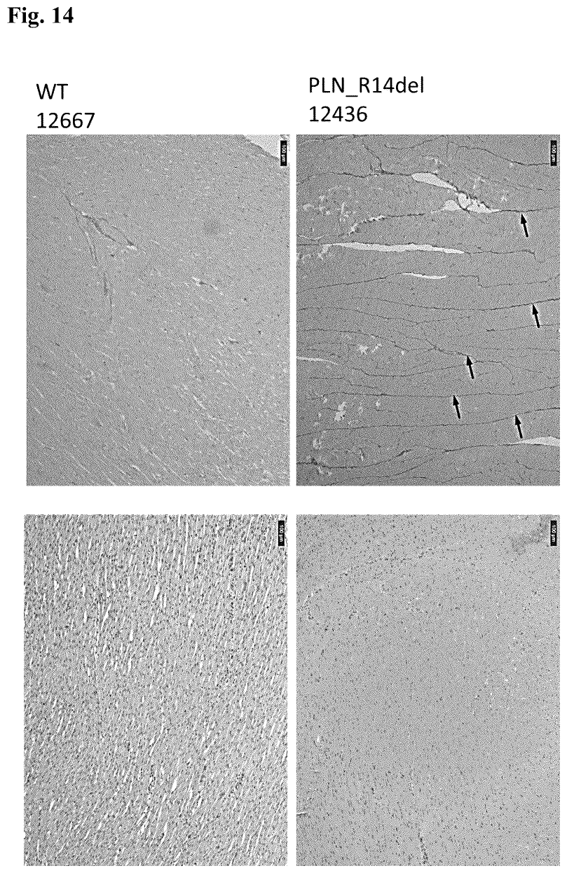

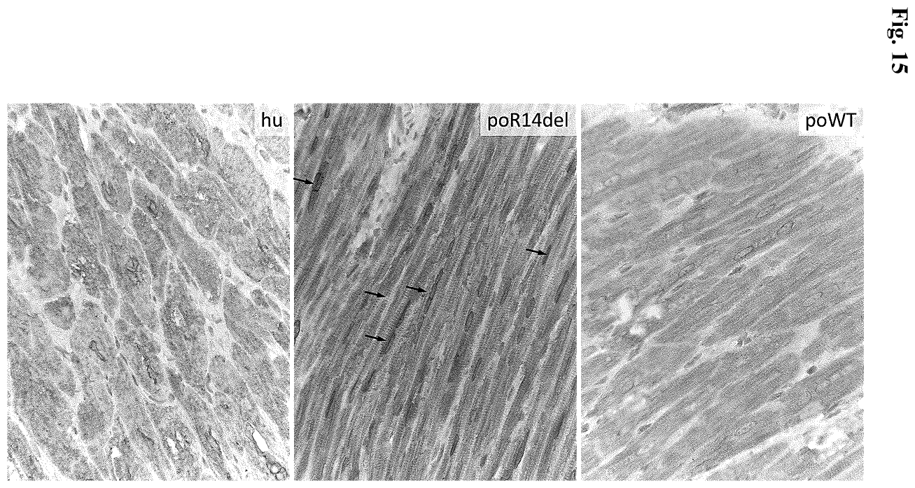

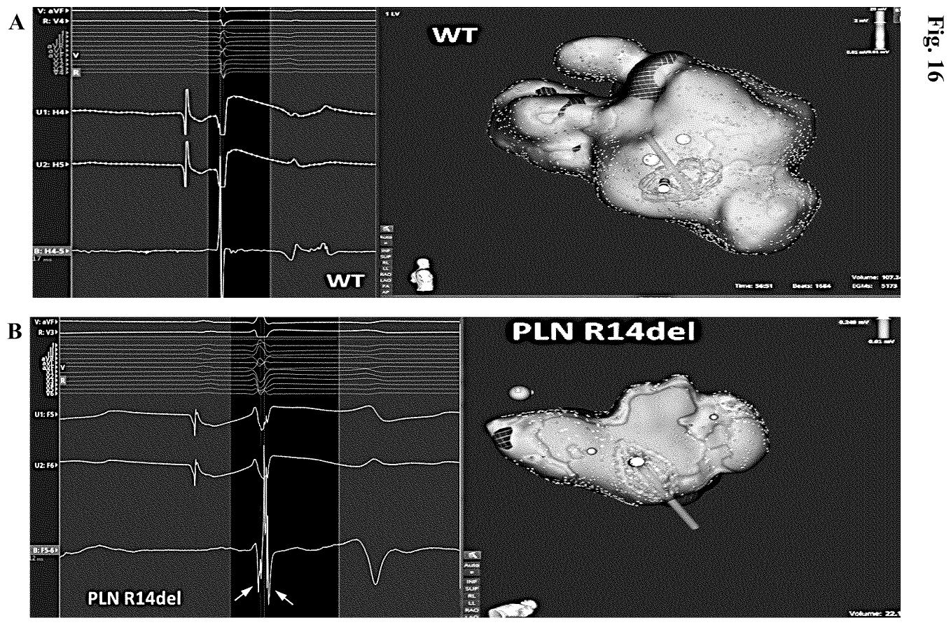

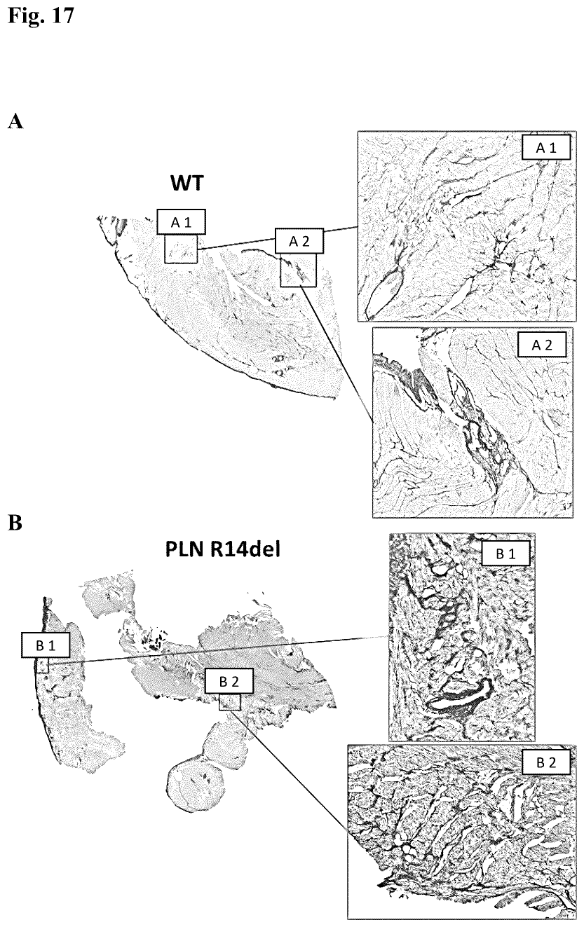

In addition to the humanization of the immediate coding region, the genetic manipulation was on purpose decided to extend to the entire PLN exon 2, which is covering a part of the 5′- and the entire 3′-UTR of the mature PLN mRNA. The intention of introducing such extended humanisation was to facilitate the future research and application of human-specific nucleic-acid therapies, and also to allow the exploitation of some more distant and specific peculiarities of PLN patients. Specifically, two single nucleotide polymorphisms (SNPs) that consistently appeared on the diseased allele of the Dutch PLN R14del patients were also on purpose included in the humanisation. In The Single Nucleotide Polymorphism Database (dbSNP; https://www.ncbi.nlm.nih.gov/snp), established by collaboration between NCBI and the National Human Genome Research Institute (NHGRI), these two SNPs are identified under ID nos. rs12198461 (T/G) and rs1051429 (C/T) and their position is shown in FIG. 4 within the human PLN genomic region (WT DNA sequence accessible e.g. under NCBI reference no. NC_000006.12) relative to the position of the deleted codon 14 (dbSNP ID no. rs397516784 and SEQ ID NO: 32: AAGAAGAGCC/AAGAGCC; deleted codon underlined). Both of these SNPs are located in the 3′-UTR of the human PLN transcript and, importantly, represent sites that discriminate the mutated allele from the intact WT PLN in the heterozygous patients, which difference can be used to the advantage of developing either allele-specific RNA-interference or gene editing approaches.

Finally, in fear that the viability or fertility of the transgenic pig model would be affected, and thus interfere its reproduction to the extent that normal breeding would no longer be possible, the transgenic PLN pig model was designed in an inducible way. This was facilitated by the fact that the PLN coding region is entirely localised to only exon 2. Hence, the genetic modification construct was designed to contain two consecutive cassettes as shown in FIG. 5. According to this design, the first cassette (“disease cassette”; marked with an asterisk in FIG. 5) contained the first transgene with the mutated human exon 2 encoding for the human PLN R14del mutant protein and was positioned downstream of a second (“rescue”) cassette containing a floxed second transgene containing the human WT exon 2 encoding for the human PLN WT protein. Upon integration of this construct under the native porcine PLN promoter by replacing the native porcine exon 2 as shown in FIG. 5A, only the second transgene would be expressed resulting in the production of the human PLN WT protein.

The expression of the human PLN WT protein was not expected to affect the health or the fertility of the transgenic animals, which would allow to maintain the transgenic population by conventional breeding in case the PLN R14del mutant protein expression would result in e.g. early mortality phenotype. However, by removal of the rescue cassette by e.g. Cre-mediated recombination, the expression of the first transgene would be induced, resulting in the production human PLN R14del mutant protein with the intention to induce the phenotype reminiscent of the human disease.

Preparation of a modified BAC vector for targeting the porcine exon 2 and performing this replacement is shown in FIG. 5B. The step-wise approach of replacing the native porcine exon 2, first with the modification construct containing both the rescue and the disease cassettes as introduced by the targeting BAC via homologous recombination, and then followed by excision of the floxed rescue cassette to achieve a humanised exon 2 locus capable of expressing the human PLN R14del mutant protein is shown in FIG. 5C. In line with this scheme, the maintenance of PLN function on the modified allele was planned to be done by a WT copy of human exon 2. Excision of the rescue cassette and thereby activation of the diseased phenotype was planned to be done by Cre-recombinase. The strategy was chosen such to facilitate a number of distinct adaptations for working with the PLN piglets and for propagating the transgenic population, depending on the phenotypical appearance of the model and in case viability issues would occur, in particular before reaching breeding maturity.

Different options and fallback positions for establishing the transgenic population based on possible genetic configurations (also termed as “constellations”) as achievable using the chosen genetic modification strategy and in the function of the associated therewith phenotypes are shown in FIG. 6.

In detail, the primary modification strategy is shown in the left panel and involves a mono-allelic replacement of the porcine PLN exon 2 with the cassette described in FIG. 5. Genomic insertion of the entire cassette was assumed to produce healthy breeding transgenic animals. Crossing of these animals with Cre-expressing transgenic pigs (second arrow in the left panel) or through introduction of Cre into one-cell embryos in vitro (ad 1), will cause the excision of the rescue cassette and the generation of a transgenic pig with the genetic constellation 2.1 (bottom left panel) and 1.1 (top right panel), respectively. Following this excision, mutant protein expression will be enabled. Under the assumption of a successful reproduction of the human-observed phenotype as caused by the dominant negative effect of the mutant protein expression, we assumed that this will result in the generation of diseased animals. Alternatively, for streamlining the procedure and generating such diseased animals at an earliest possible time point (genetic constellation 2.1, bottom left panel), i.e. already in the founder generation (F), transfection of pig primary cells with a Cre-encoding mRNA will be tried before SCNT.

In case transgenic animals with genetic constellation 2.1, which are obtained following the excision of the rescue cassette, develop properly and reach fertility, these animals will be reproduced by conventional breeding (ad2) and the maintenance of an independent breeding population will not be necessary. However, in case the full phenotypical manifestation and reproduction of the disease observed in human patients requires an interaction between two human alleles, one being the healthy one and the second being the mutated one (ad3), the human WT copy will be fixed in the transgenic animal's genome during in vitro fertilization through excision of the downstream elements by e.g. CRISPR/Cas (arrows shown in genetic constellation 3.1 in FIG. 6). Animals with a fixed human WT allele (genetic constellation 3.2) can then be mated with the animals comprising the basic modification, and the desired WT/R14del constellation (genetic constellation 3.3.) can then be induced by Cre recombinase.

For testing patient specific nucleic acid treatments (ad4), the porcine WT copy can be introduced by genetic modification into a cell line comprising the initially generated full modification (genetic constellation 4.1). Diseased animals can be generated using Cre (genetic constellation 4.2). The benefit of a non-patient-specific WT allele in (ad4) to the fixation of a WT copy in the existing modification (ad3) is counterbalanced by the higher effort of the modification process and additional SCNT experiments.

Although, obtaining a transgenic pig model with genetic constellation 3.3, i.e. containing two knocked-in human exons 2 in place of porcine exons 2, one encoding for a FL WT human PLN protein (represented by SEQ ID NO: 1, cf. Table 1) and the other one for a FL R14del mutant protein (SEQ ID NO: 1, cf. Table 1), is eventually intended, breeding of such animal may require establishment of an initial breeding population, and hence time before (and if) the transgenic pigs reach puberty and are confirmed to be fertile. Consequently, it was decided that a transgenic pig with the genetic constellation 1.1, i.e. comprising one remaining porcine PLN exon 2 generating FL WT porcine PLN, and one human PLN mutant exon 2 generating a FL human PLN R14del mutant protein, will be generated first, to first assess its 10 viability, fertility, and potential reconstruction of the diseased phenotype as observed in the affected human subjects, preferably to be observed in the pig heart and/or its cells.

TABLE 1

| # | Desription | Sequence |
|---|---|---|
| SEQ ID NO: 1 | Human PLN protein | MEKVQYLTRSAIRRASTIEMPQQARQKLQNLFI |
| | (FL, WT, RefSeq no. NP_002658.1) | NFCLILICLLLICIIVMLL |
| SEQ ID NO: 2 | Human PLN R14del | MEKVQYLTRSAIRASTIEMPQQARQKLQNLFIN |
| | mutant protein (FL) | FCLILICLLLICIIVMLL |
| SEQ ID NO: 3 | Primer BAC sequencing #1: | CACACCATGACCGTCTCATT |
| SEQ ID NO: 4 | Primer BAC sequencing #2: | TGCTGAGGAAGATGAATTAGTG |
| SEQ ID NO: 5 | Primer BAC sequencing #3: | CCCAAGTCCAGACACACAAG |
| SEQ ID NO: 6 | Primer BAC sequencing #4: | GACAATAGCAGGCATGCTG |
| SEQ ID NO: 7 | Primer BAC sequencing #5: | GTGGATGTGGAATGTGTGC |
| SEQ ID NO: 8 | Primer qPCR PLN forward: | GGTTTCACACTGATAGAGGGATT |
| SEQ ID NO: 9 | Primer qPCR PLN reverse: | ACCAGTAAAGCAGTTGTCTCAT |
| SEQ ID NO: 10 | Primer qPCR USH forward: | CTTCTTGGAGCAGATGGGATAAA |
| SEQ ID NO: 11 | Primer qPCR USH reverse: | GAATGCAGATTTCTGGTTTCCAC |
| SEQ ID NO: 12 | Primer qPCR NG forward: | GAGGCTTCACTTGTTAAGGG |
| SEQ ID NO: 13 | Primer qPCR NG reverse: | CTGAGATCAGGGTAGACATAC |
| SEQ ID NO: 14 | Primer end-point PCR #1 forward: | AAGGAATGGCAAACATTTCA |
| SEQ ID NO: 15 | Primer end-point PCR #1 reverse: | GGAGTCAGTGGGCTATTTCA |
| SEQ ID NO: 16 | Primer end-point PCR #2 forward: | AAGGAATGGCAAACATTTCA |
| SEQ ID NO: 17 | Primer end-point PCR #2 reverse: | CACACCATGACCGTCTCATT |
| SEQ ID NO: 18 | Primer end-point PCR #3 forward: | TGCTGAGGAAGATGAATTAGTG |
| SEQ ID NO: 19 | Primer end-point PCR #3 reverse: | TGGGATAGAAATTTGTGAGCCA |
| SEQ ID NO: 20 | Primer end-point PCR #4 forward: | CACTCGCTCAGCTATAAGAAGA |
| SEQ ID NO: 21 | Primer end-point PCR #4 reverse: | TGGGATAGAAATTTGTGAGCCA |
| SEQ ID NO: 22 | Primer end-point PCR #5 forward: | CCCAAGTCCAGACACACAAG |
| SEQ ID NO: 23 | Primer end-point PCR #5 reverse: | GTGGATGTGGAATGTGTGC |
| SEQ ID NO: 24 | Primer end-point PCR #6 forward: | GACAATAGCAGGCATGCTG |
| SEQ ID NO: 25 | Primer end-point PCR #6 reverse: | CACACCATGACCGTCTCATT |
| SEQ ID NO: 26 | Primer porcine RT-PCR forward: | ACACCGATAAGACTTCATACAACT |
| SEQ ID NO: 27 | Primer porcine RT-PCR reverse: | TGTAGGTTCTGATACCACTAGAGA |
| SEQ ID NO: 28 | Primer human RT-PCR forward: | CTGTGATCATCATAAACAGCCAAG |
| SEQ ID NO: 29 | Primer human RT-PCR reverse: | AAAGACAGCAGATGTGAGGAG |

### Generation of the PLN Transgenic Pig Model

Human PLN exon 2 DNA molecules encoding for the human WT and the human R14del mutant PLN proteins and including the patient specific SNPs in addition to the WT or mutated coding regions, respectively, were commercially synthesized. Then, they were combined with a neomycin selection cassette by classical plasmid cloning into a modification plasmid (represented by SEQ ID NO: 30, shown below) for replacing porcine PLN exon 2 with a rescue cassette construct, as shown in FIG. 5.

Sequence (SEQ ID NO. 30) and elements of the modification vector:

| Element | Position |
|---|---|
| 5′-homologous arm | 1-300 |
| loxP-site | 328-361 |
| PLN rescue cassette | 362-4260 |
| Including human PLN WT exon 2 | 869-3759 |
| Including human PLN WT coding sequence | 966-1124 |
| Neomycin resistance cassette | 4266-5992 |
| loxP-site | 5993-6026 |
| PLN rescue cassette | 6033-9921 |
| Including human PLN WT exon 2 | 6533-9420 |
| Including human PLN WT coding sequence | 6630-6785 |
| 3′-homologous arm | 9928-10227 |

| | |
|---|---|
| 1 | gcttcagaaa atctatacta aaatctcaaa acatgtagat gtgaataaat gtaaactctt |
| 61 | caaatgcgct ttatttttac atcttattcc agaaatgctc agaaaTaaaa agaatatctc |
| 121 | tttgaaacag tatctaatgt catacttaaa tgcactcatt tctttttatt agattgctct |
| 181 | ggagccatta atacactaat gttcacaaca attatgctca tagagaaacg gtcttaatga |
| 241 | aactgtatgt aagaacttgc ctaagggaac agcccaaaac aagctgcccc ctaaactaaa |
| 301 | atgcatgaca ataaacgcta gctggcaATA ACTTCGTATA ATGTATGCTA TACGAAGTTA |
| 361 | Tgggatcctt ggggtcttta ttgagaagtt tggtgatgtt tttgtgacca gaaatatgct |
| 421 | ataggaactt aactcttgtt tacatcaatc agcctagggt aaaaatggtt ttgttatatg |
| 481 | ttgttttgcg taaagtcatg gtttccagga acttatggaa gacactgagg attactccaa |
| 541 | tacttgtagt aaatttggga gaaaaaggca aggactaaag ttactactcc taacacaaat |
| 601 | gagacggtca tggtgtgccc catcaaaaaa ggagagaaag agagacagac acatagaaac |
| 661 | acgtgcacat acacacacac acacacacac acacacacac acacagagag agagagagag |
| 721 | agagagagag agggagagag actatagaaa catgatgtta taaataacaa tagtgctgag |
| 781 | gaagatgaat tagtgtaaaa ttgtattttt tgttctgagg ataggttaca tagatgattc |
| 841 | taatccattt attattttta cattccaggc tacctaaaag aagacagtta tctcatattt |
| 901 | ggctgccagc tttttatctt tctctcgacc acttaaaact tcagacttcc tgtcctgctg |
| 961 | gtatcATGGA GAAAGTCCAA TACCTCACTC GCTCAGCTAT AAGAAGAGCC TCAACCATTG |
| 1021 | AAATGCCTCA ACAAGCACGT CAAAAGCTAC AGAATCTATT TATCAATTTC TGTCTCATCT |
| 1081 | TAATATGTCT CTTGCTGATC TGTATCATCG TGATGCTTCT CTGAagttct gctacaacct |
| 1141 | ctagatctgc agcttgccac atcagcttaa aatctgtcat cccatgcaga caggaaaaca |
| 1201 | atattgtata acagaccact tcctgagtag aagagtttct ttgtgaaaag gtcaagatta |
| 1261 | agactaaaac ttattgttac catatgtatt catctgttgg atcttgtaaa catgaaaagg |
| 1321 | gctttatttt caaaaattaa cttcaaaata agtgtataaa atgcaactgt tgatttcctc |
| 1381 | aacatggctc acaaatttct atcccaaatc ttttctgaag atgaagagtt tagttttaaa |
| 1441 | actgcactgc caacaagttc acttcatata taaagcatta tttttactct tttgaggtga |
| 1501 | atataattta tattacaatg gaaaagcttc tttaatacta agtatttttc aggtcttcac |
| 1561 | caagtatcaa agtaataaca caaatgaagt gtcattattc aaaatagtcc actgactcct |
| 1621 | cacatctgtt atcttattat aaagaactat ttgtagtaac tatcagaatc tacattctaa |
| 1681 | aacagaaatt gtattttttc tatgccacat taacatcttt taaagttgat gagaatcaag |
| 1741 | tatggaaaag taaggccata ctcttacata ataaaattcc ttttaagtaa ttttttcaaa |
| 1801 | gaatcacaga attctagtac atgtaggtaa atcataaatc tgttctaaga catatgatca |
| 1861 | acagatgaga actggtggtt aatatgtgac agtgagatta gtcatatcac taatatacta |
| 1921 | acaacagaat ctaatcttca tttaaggcac tgtagtgaat tatctgagct agagttacct |
| 1981 | agcttaccat actatatctt tggaatcatg aaaccttaag acttcagaat gattttgcag |
| 2041 | gttgtcttcc attccagcct aacatccaat gcaggcaagg aaaataaaag atttccagtg |
| 2101 | acagaaaaat atattatctc aagtattttt taaaaatata tgaattctct ctccaaatat |
| 2161 | taactaatta ttagattata ttttgaaatg aacttgttgg cccatctatt acatctacag |
| 2221 | ctgacccttg aacatggggg ttaggggagc tgacaattcg tgggtccgca aaatcttaac |
| 2281 | tacctaatag cctactattg accataaacc ttactgataa cataaacagt aaattaacac |
| 2341 | atattttgcg tgttatatgt attatacact atattcctac aataaagtaa gctagagaaa |
| 2401 | atgttattta gaaaatcata agaaagagaa aatatattta ctattcatta aatggaagtg |
| 2461 | ggtcaacata aaagtcttca ttctcattgt cttcacattg agtaggcaga ggaggagaaa |
| 2521 | gatggggagg aagagaaggc gttggtcttg cagtcttgtc ttaggggtgt ggggagtggg |
| 2581 | ggaaagaata ttcatgtata agtggaccct tgcaattcaa gcccttgttg ttcaagggtc |
| 2641 | aactgtaata ggatatagct atttttcttc ctctatcaac caaatggtaa gcatctattt |
| 2701 | tgtagtccac tctactgagc taaattatag atccagctat gctatttata attattttct |
| 2761 | tgatgaataa attttcaatt tctcctctga ccatttcaga acatcttcca ataactcata |
| 2821 | aaacaactga agtaaaattg agtgctggaa aatatattca ccaaactttg gtaatttaag |
| 2881 | ttgactaaag tttaaaatta agtctaaaat agtttacacc tatactgcat aatccaacaa |
| 2941 | ttttaatttc agttgaagac atgttactaa tataactatt attaaaagag tagaggatgt |
| 3001 | gtaattaacc atatcttcta aaacatggtt actaaaagaa tatgtaacat caatattgac |
| 3061 | cttggtttct tacacaagtg ttgctaactc aatagtgaag gagacactat taaattttct |
| 3121 | gaacccatga gagatactag agatggggag tggaaagtgt ttggttcagg gatatctgaa |
| 3181 | gaacagaagg gcagagattt cttaagtgac gcctcatcta caagctggaa attcctaaaa |
| 3241 | acaagtagaa agcttataaa caacaggtga tacactcacc tcactggttt tagtaaatta |
| 3301 | ccaatacaga aagtatccct agtcttaaaa acaagtggaa aatttgaact gattagtcat |
| 3361 | attcctttga ttacactgtt tgttacaata tttttctcag taaacagaaa taactaattt |
| 3421 | ttttgttctt cattctttga tagaaattaa aatcttattc tgtgaggatt acagaatact |
| 3481 | ataactcaaa ttataaagta gaataaactc tttaaataat tattcttcat cataaagtgt |
| 3541 | aaagaataag atataagaaa acaatttatt tttaaaattt aatatactaa atgctcaaat |
| 3601 | atgttctact atagaataag ttcttatctt aatttacagg gcactaaaaa caattttaaa |
| 3661 | atgcttaatg ttgcctttta tattttaatt ggttaagaat atatatttgt ttaatgcaaa |
| 3721 | tcagaatcac tatattaaaa tgaatgttct tgaaaactca gtggggctgc tctataatac |
| 3781 | acttacaatt gatactaacg ataaaacgct ctaattatag ttttaatttt ataggggttt |
| 3841 | ttttggtgac tttccccaaa taaaacaaaa tcaagacgcc caagtccaga cacacaagag |
| 3901 | ggcagacgta tataagataa caaattgcta ctacatagcg attatttatc ttttgattat |
| 3961 | ttttgagact tctggcaaag taggactata ataaattttc agaaaagatc tgctcttctg |
| 4021 | ctaagtaaca gcacacttca aaatgtccag aaattttatg ctaatattac atagaaaact |
| 4081 | tgacagatga aaaaagtgaa acacattact aattttattc attaaaacag gtatataaaa |
| 4141 | gtcctcttta actacctaat gaatgtgtca tatcctaata tagggagtgt catagtgttc |
| 4201 | acatctttga atatataaat tatctcaaac agttaaaaaa ttaaagccag aacagggtac |
| 4261 | actagcttgg ctgcaggtcg tcgaaattct accgggtagg ggaggcgctt ttcccaaggc |
| 4321 | agtctggagc atgcgcttta gcagccccgc tgggcacttg gcgctacaca agtggcctct |
| 4381 | ggcctcgcac acattccaca tccaccggta ggcgccaacc ggctccgttc tttggtggcc |
| 4441 | ccttcgcgcc accttctact cctcccctag tcaggaagtt cccccccgcc ccgcagctcg |
| 4501 | cgtcgtgcag gacgtgacaa atggaagtag cacgtctcac tagtctcgtg cagatggaca |
| 4561 | gcaccgctga gcaatggaag cgggtaggcc tttggggcag cggccaatag cagctttgct |
| 4621 | ccttcgcttt ctgggctcag aggctgggaa ggggtgggtc cgggggcggg ctcaggggcg |
| 4681 | ggctcagggg cggggcgggc gcccgaaggt cctccggagg cccggcattc tgcacgcttc |
| 4741 | aaaagcgcac gtctgccgcg ctgttctcct cttcctcatc tccgggcctt tcgacctgca |
| 4801 | gcctgttgac aattaatcat cggcatagta tatcggcata gtataatacg acaaggtgag |
| 4861 | gaactaaacc atgggatcgg ccattgaaca agatggattg cacgcaggtt ctccggccgc |
| 4921 | ttgggtggag aggctattcg gctatgactg ggcacaacag acaatcggct gctctgatgc |
| 4981 | cgccgtgttc cggctgtcag cgcaggggcg cccggttctt tttgtcaaga ccgacctgtc |
| 5041 | cggtgccctg aatgaactgc aggacgaggc agcgcggcta tcgtggctgg ccacgacggg |
| 5101 | cgttccttgc gcagctgtgc tcgacgttgt cactgaagcg ggaagggact ggctgctatt |
| 5161 | gggcgaagtg ccggggcagg atctcctgtc atctcacctt gctcctgccg agaaagtatc |
| 5221 | catcatggct gatgcaatgc ggcggctgca tacgcttgat ccggctacct gcccattcga |
| 5281 | ccaccaagcg aaacatcgca tcgagcgagc acgtactcgg atggaagccg gtcttgtcga |
| 5341 | tcaggatgat ctggacgaag agcatcaggg gctcgcgcca gccgaactgt tcgccaggct |
| 5401 | caaggcgcgc atgcccgacg gcgatgatct cgtcgtgacc catggcgatg cctgcttgcc |
| 5461 | gaatatcatg gtggaaaatg gccgcttttc tggattcatc gactgtggcc ggctgggtgt |
| 5521 | ggcggaccgc tatcaggaca tagcgttggc tacccgtgat attgctgaag agcttggcgg |
| 5581 | cgaatgggct gaccgcttcc tcgtgcttta cggtatcgcc gctcccgatt cgcagcgcat |
| 5641 | cgccttctat cgccttcttg acgagttctt ctgaggggat caattctcta gagctcgctg |
| 5701 | atcagcctcg actgtgcctt ctagttgcca gccatctgtt gtttgcccct cccccgtgcc |
| 5761 | ttccttgacc ctggaaggtg ccactcccac tgtcctttcc taataaaatg aggaaattgc |
| 5821 | atcgcattgt ctgagtaggt gtcattctat tctggggggt ggggtggggc aggacagcaa |
| 5881 | gggggaggat tgggaagaca atagcaggca tgctggggat gcggtgggct ctatggcttc |
| 5941 | tgaggcggaa agaaccagct ggggctcgac tagagcttgc ggaaccctta atATAACTTC |
| 6001 | GTATAATGTA TGCTATACGA AGTTATggat ccttggggtc tttattgaga agtttggtga |
| 6061 | tgtttttgtg accagaaata tgctatagga acttaactct tgtttacatc aatcagccta |
| 6121 | gggtaaaaat ggttttgtta tatgttgttt tgcgtaaagt catggtttcc aggaacttat |
| 6181 | ggaagacact gaggattact ccaatacttg tagtaaattt gggagaaaaa ggcaaggact |
| 6241 | aaagttacta ctcctaacac aaatgagacg gtcatggtgt gccccatcaa aaaaggagag |
| 6301 | aaagagagac agacacatag aaacacgtgc acatacacac acacacacac acacacacac |
| 6361 | acacacacag agagagagag agagagagag agagagggag agagactata gaaacatgat |
| 6421 | gttataaata acaatagtgc tgaggaagat gaattagtgt aaaattgtat tttttgttct |
| 6481 | gaggataggt tacatagatg attctaatcc atttattatt tttacattcc aggctaccta |
| 6541 | aaagaagaca gttatctcat atttggctgc cagcttttta tctttctctc gaccacttaa |
| 6601 | aacttcagac ttcctgtcct gctggtatcA TGGAGAAAGT CCAATACCTC ACTCGCTCAG |
| 6661 | CTATAAGA-- -GCCTCAACC ATTGAAATGC CTCAACAAGC ACGTCAAAAG CTACAGAATC |
| 6721 | TATTTATCAA TTTCTGTCTC ATCTTAATAT GTCTCTTGCT GATCTGTATC ATCGTGATGC |
| 6781 | TTCTCTGAag ttctgctaca acctctagat ctgcagcttg ccacatcagc ttaaaatctg |
| 6841 | tcatcccatg cagacaggaa aacaatattg tataacagac cacttcctga gtagaagagt |
| 6901 | ttctttgtga aaaggtcaag attaagacta aaacttattg ttaccatatg tattcatctg |
| 6961 | ttggatcttg taaacatgaa aagggcttta ttttcaaaaa ttaacttcaa aataagtgta |
| 7021 | taaaatgcaa ctgttgattt cctcaacatg gctcacaaat ttctatccca aatcttttct |
| 7081 | gaagatgaag agtttagttt taaaactgca ctgccaacaa gttcacttca tatataaagc |
| 7141 | attattttta ctcttttgag gtgaatataa tttatattac aatggaaaag cttctttaat |
| 7201 | actaagtatt tttcaggtct tcaccaagta tcaaagtaat aacacaaatg aagtgtcatt |
| 7261 | attcaaaata gtccactgac tcctcacatc tgttatctta ttataaagaa ctatttgtag |
| 7321 | taactatcag aatctacatt ctaaaacaga aattgtattt tttctatgcc acattaacat |
| 7381 | cttttaaagt tgatgagaat caagtatgga aaagtaaggc catactctta cataataaaa |
| 7441 | ttccttttaa gtaatttttt caaagaatca cagaattcta gtacatgtag gtaaatcata |
| 7501 | aatctgttct aagacatatg atcaacagat gagaactggt ggttaatatg tgacagtgag |
| 7561 | attagtcata tcactaatat actaacaaca gaatctaatc ttcatttaag gcactgtagt |
| 7621 | gaattatctg agctagagtt acctagctta ccatactata tctttggaat catgaaacct |
| 7681 | taagacttca gaatgatttt gcaggttgtc ttccattcca gcctaacatc caatgcaggc |
| 7741 | aaggaaaata aaagatttcc agtgacagaa aaatatatta tctcaagtat tttttaaaaa |
| 7801 | tatatgaatt ctctctccaa atattaacta attattagat tatattttga aatgaacttg |
| 7861 | ttggcccatc tattacatct acagctgacc cttgaacatg ggggttaggg gagctgacaa |
| 7921 | ttcgtgggtc cgcaaaatct taactaccta atagcctact attgaccata aaccttactg |
| 7981 | ataacataaa cagtaaatta acacatattt tgcgtgttat atgtattata cactatattc |
| 8041 | ctacaataaa gtaagctaga gaaaatgtta tttagaaaat cataagaaag agaaaatata |
| 8101 | tttactattc attaaatgga agtgggtcaa cataaaagtc ttcattctca ttgtcttcac |
| 8161 | attgagtagg cagaggagga gaaagatggg gaggaagaga aggcgttggt cttgcagtct |
| 8221 | tgtcttaggg gtgtggggag tgggggaaag aatattcatg tataagtgga cccttgcaat |
| 8281 | tcaagccctt gttgttcaag ggtcaactgt aataggatat agctattttt cttcctctat |
| 8341 | caaccaaatg gtaagcatct attttgtagt ccactctact gagctaaatt atagatccag |
| 8401 | ctatgctatt tataattatt ttcttgatga ataaattttc aatttctcct ctgaccattt |
| 8461 | cagaacatct tccaataact cataaaacaa ctgaagtaaa attgagtgct ggaaaatata |
| 8521 | ttcaccaaac tttggtaatt taagttgact aaagtttaaa attaagtcta aaatagttta |
| 8581 | cacctatact gcataatcca acaattttaa tttcagttga agacatgtta ctaatataac |
| 8641 | tattattaaa agagtagagg atgtgtaatt aaccatatct tctaaaacat ggttactaaa |
| 8701 | agaatatgta acatcaatat tgaccttggt ttcttacaca agtgttgcta actcaatagt |
| 8761 | gaaggagaca ctattaaatt ttctgaaccc atgagagata ctagagatgg ggagtggaaa |
| 8821 | gtgtttggtt cagggatatc tgaagaacag aagggcagag atttcttaag tgacgcctca |
| 8881 | tctacaagct ggaaattcct aaaaacaagt agaaagctta taaacaacag gtgatacact |
| 8941 | cacctcactg gttttagtaa attaccaata cagaaagtat ccctagtctt aaaaacaagt |
| 9001 | ggaaaatttg aactgattag tcatattcct ttgattacac tgtttgttac aatatttttc |
| 9061 | tcagtaaaca gaaataacta atttttttgt tcttcattct ttgatagaaa ttaaaatctt |
| 9121 | attctgtgag gattacagaa tactataact caaattataa agtagaataa actctttaaa |
| 9181 | taattattct tcatcataaa gtgtaaagaa taagatataa gaaaacaatt tatttttaaa |
| 9241 | atttaatata ctaaatgctc aaatatgttc tactatagaa taagttctta tcttaattta |
| 9301 | cagggcacta aaaacaattt taaaatgctt aatgttgcct tttatatttt aattggttaa |
| 9361 | gaatatatat ttgtttaatg caaatcagaa tcactatatt aaaatgaatg ttcttgaaaa |
| 9421 | ctcagtgggg ctgctctata atacacttac aattgatact aacgataaaa cgctctaatt |
| 9481 | atagttttaa ttttataggg gtttttttgg tgactttccc caaataaaac aaaatcaaga |
| 9541 | cgcccaagtc cagacacaca agagggcaga cgtatataag ataacaaatt gctactacat |
| 9601 | agcgattatt tatcttttga ttatttttga gacttctggc aaagtaggac tataataaat |
| 9661 | tttcagaaaa gatctgctct tctgctaagt aacagcacac ttcaaaatgt ccagaaattt |
| 9721 | tatgctaata ttacatagaa aacttgacag atgaaaaaag tgaaacacat tactaatttt |
| 9781 | attcattaaa acaggtatat aaaagtcctc tttaactacc taatgaatgt gtcatatcct |
| 9841 | aatataggga gtgtcatagt gttcacatct ttgaatatat aaattatctc aaacagttaa |
| 9901 | aaaattaaag ccagaacagg gtacactagt aaaaataaaa ataagaaaac tttcttttca |
| 9961 | gaggatgtag tagtatacat aaactaccct cacattaaaa atagaacctt ttcaaattga |
| 10021 | aactacaatg aggtaccaac tcacaccagt cagaatggcc atcattagta aatccacaaa |
| 10081 | aaataaatgc tgaagagggg gtggagaaaa gggaaccctc ctaaactgct ggtgggaatg |
| 10141 | taaattggta taactactat ggaaagaagt atggaggttc ctcagaaaat taaatataga |
| 10201 | actaccctat gatccagcaa tcccactcct |

DNA encoding for the human WT and the R14del mutant PLN proteins, as used for the cloning, were practically identical with the obvious exception of the lack of the Arg codon in the mutated DNA, and further a minor indel variation downstream of the lox sites. Namely, while in the WT DNA copy a single additional guanine (“G”) was between the lox and the adjacent BamHI site, this nucleotide was missing in the mutated exon 2 copy encoding for the R14del mutant PLN protein. The position of this single nucleotide sequence variation is marked with a pin in FIGS. 7 and 5A and can be exploited for genotyping once the modification is successfully introduced in the transgenic animals.

The modification plasmid (SEQ ID NO: 30) was introduced by bacterial recombineering into the bacterial artificial chromosome (BAC) CH242-318M5, covering the porcine PLN locus, as shown in FIG. 5 (Warming et al., 2005). After the recombineering, the correct modification and integrity of BAC clones was verified by a combination of a discriminative restriction digest with Ncol enzyme and an end-point PCR-. The NcoI-digested-BAC clones were verified by gel electrophoresis for the presence of a band pattern as predicted in silico. A correctly humanized BAC would show two unique bands at 8753 bp and 8419 bp as marked in lane 2 and 3 of the gel as shown in the bottom left panel in FIG. 7. Transitions from the modification cassette into the original BAC sequence were confirmed by Sanger sequencing. The arrows in the upper panel of FIG. 7 indicate approximate positions and orientations of the sequencing primers #1-#5 (SEQ ID NOs: 3-7, respectively, cf. Table 1).

The successfully verified targeting vectors transcribing gRNAs BACI and BAC3 or BAC2 were used for co-nucleofection of primary pig kidney cells with a plasmid encoding Cas9 (represented by SEQ ID NO: 31; additional details provided below).

Sequence (SEQ ID NO. 31) and elements of Cas9 vector
(acc._Mali P, Yang L, Esvelt KM, Aach J, Guell M, DiCarlo JE, Norville JE, Church
GM.RNA-guided human genome engineering via Cas9. Science. 2013 Feb. 15;339(6121):
823-6. doi: 10.1126/science. 1232033. PMID:23287722)

| Element | Position |
|---|---|
| huCMV enhancer | 114-430 |
| huCMV promoter | 431-747 |
| Cas9 coding region | 748-4887 |

| | |
|---|---|
| 1 | GTTAGGCGTT TTGCGCTGCT TCGCGATGTA CGGGCCAGAT ATACGCGTTG ACATTGATTA |
| 61 | TTGACTAGTT ATTAATAGTA ATCAATTACG GGGTCATTAG TTCATAGCCC ATATATGGAG |
| 121 | TTCCGCGTTA CATAACTTAC GGTAAATGGC CCGCCTGGCT GACCGCCCAA CGACCCCCGC |
| 181 | CCATTGACGT CAATAATGAC GTATGTTCCC ATAGTAACGC CAATAGGGAC TTTCCATTGA |
| 241 | CGTCAATGGG TGGAGTATTT ACGGTAAACT GCCCACTTGG CAGTACATCA AGTGTATCAT |
| 301 | ATGCCAAGTA CGCCCCCTAT TGACGTCAAT GACGGTAAAT GGCCCGCCTG GCATTATGCC |
| 361 | CAGTACATGA CCTTATGGGA CTTTCCTACT TGGCAGTACA TCTACGTATT AGTCATCGCT |
| 421 | ATTACCATGG TGATGCGGTT TTGGCAGTAC ATCAATGGGC GTGGATAGCG GTTTGACTCA |
| 481 | CGGGGATTTC CAAGTCTCCA CCCCATTGAC GTCAATGGGA GTTTGTTTTG GCACCAAAAT |
| 541 | CAACGGGACT TTCCAAAATG TCGTAACAAC TCCGCCCCAT TGACGCAAAT GGGcGGTAGG |
| 601 | CGTGTACGGT GGGAGGTCTA TATAAGCAGA GCTCGTTTAG TGAACCGTCA GATCGCCTGG |
| 661 | AGACGCCATC CACGCTGTTT TGACCTCCAT AGAAGACACC GGGACCGATC CAGCCTCCGG |
| 721 | ACTCTAGAGG ATCGAACCCT TgccaccATG GACAAGAAGT ACTCCATTGG GCTCGATATC |
| 781 | GGCACAAACA GCGTCGGCTG GGCCGTCATT ACGGACGAGT ACAAGGTGCC GAGCAAAAAA |
| 841 | TTCAAAGTTC TGGGCAATAC CGATCGCCAC AGCATAAAGA AGAACCTCAT TGGCGCCCTC |
| 901 | CTGTTCGACT CCGGGGAGAC GGCCGAAGCC ACGCGGCTCA AAAGAACAGC ACGGCGCAGA |
| 961 | TATACCCGCA GAAAGAATCG GATCTGCTAC CTGCAGGAGA TCTTTAGTAA TGAGATGGCT |
| 1021 | AAGGTGGATG ACTCTTTCTT CCATAGGCTG GAGGAGTCCT TTTTGGTGGA GGAGGATAAA |
| 1081 | AAGCACGAGC GCCACCCAAT CTTTGGCAAT ATCGTGGACG AGGTGGCGTA CCATGAAAAG |
| 1141 | TACCCAACCA TATATCATCT GAGGAAGAAG CTTGTAGACA GTACTGATAA GGCTGACTTG |
| 1201 | CGGTTGATCT ATCTCGCGCT GGCGCATATG ATCAAATTTC GGGGACACTT CCTCATCGAG |
| 1261 | GGGGACCTGA ACCCAGACAA CAGCGATGTC GACAAACTCT TTATCCAACT GGTTCAGACT |
| 1321 | TACAATCAGC TTTTCGAAGA GAACCCGATC AACGCATCCG GAGTTGACGC CAAAGCAATC |
| 1381 | CTGAGCGCTA GGCTGTCCAA ATCCCGGCGG CTCGAAAACC TCATCGCACA GCTCCCTGGG |
| 1441 | GAGAAGAAGA ACGGCCTGTT TGGTAATCTT ATCGCCCTGT CACTCGGGCT GACCCCCAAC |
| 1501 | TTTAAATCTA ACTTCGACCT GGCCGAAGAT GCCAAGCTTC AACTGAGCAA AGACACCTAC |
| 1561 | GATGATGATC TCGACAATCT GCTGGCCCAG ATCGGCGACC AGTACGCAGA CCTTTTTTTG |
| 1621 | GCGGCAAAGA ACCTGTCAGA CGCCATTCTG CTGAGTGATA TTCTGCGAGT GAACACGGAG |
| 1681 | ATCACCAAAG CTCCGCTGAG CGCTAGTATG ATCAAGCGCT ATGATGAGCA CCACCAAGAC |
| 1741 | TTGACTTTGC TGAAGGCCCT TGTCAGACAG CAACTGCCTG AGAAGTACAA GGAAATTTTC |
| 1801 | TTCGATCAGT CTAAAAATGG CTACGCCGGA TACATTGACG GCGGAGCAAG CCAGGAGGAA |
| 1861 | TTTTACAAAT TTATTAAGCC CATCTTGGAA AAAATGGACG GCACCGAGGA GCTGCTGGTA |
| 1921 | AAGCTTAACA GAGAAGATCT GTTGCGCAAA CAGCGCACTT TCGACAATGG AAGCATCCCC |
| 1981 | CACCAGATTC ACCTGGGCGA ACTGCACGCT ATCCTCAGGC GGCAAGAGGA TTTCTACCCC |
| 2041 | TTTTTGAAAG ATAACAGGGA AAAGATTGAG AAAATCCTCA CATTTCGGAT ACCCTACTAT |
| 2101 | GTAGGCCCCC TCGCCCGGGG AAATTCCAGA TTCGCGTGGA TGACTCGCAA ATCAGAAGAG |
| 2161 | ACCATCACTC CCTGGAACTT CGAGGAAGTC GTGGATAAGG GGGCCTCTGC CCAGTCCTTC |
| 2221 | ATCGAAAGGA TGACTAACTT TGATAAAAAT CTGCCTAACG AAAAGGTGCT TCCTAAACAC |
| 2281 | TCTCTGCTGT ACGAGTACTT CACAGTTTAT AACGAGCTCA CCAAGGTCAA ATACGTCACA |
| 2341 | GAAGGGATGA GAAAGCCAGC ATTCCTGTCT GGAGAGCAGA AGAAAGCTAT CGTGGACCTC |
| 2401 | CTCTTCAAGA CGAACCGGAA AGTTACCGTG AAACAGCTCA AAGAAGACTA TTTCAAAAAG |
| 2461 | ATTGAATGTT TCGACTCTGT TGAAATCAGC GGAGTGGAGG ATCGCTTCAA CGCATCCCTG |
| 2521 | GGAACGTATC ACGATCTCCT GAAAATCATT AAAGACAAGG ACTTCCTGGA CAATGAGGAG |
| 2581 | AACGAGGACA TTCTTGAGGA CATTGTCCTC ACCCTTACGT TGTTTGAAGA TAGGGAGATG |
| 2641 | ATTGAAGAAC GCTTGAAAAC TTACGCTCAT CTCTTCGACG ACAAAGTCAT GAAACAGCTC |
| 2701 | AAGAGGCGCC GATATACAGG ATGGGGGGGG CTGTCAAGAA AACTGATCAA TGGGATCCGA |
| 2761 | GACAAGCAGA GTGGAAAGAC AATCCTGGAT TTTCTTAAGT CCGATGGATT TGCCAACCGG |
| 2821 | AACTTCATGC AGTTGATCCA TGATGACTCT CTCACCTTTA AGGAGGACAT CCAGAAAGCA |
| 2881 | CAAGTTTCTG GCCAGGGGGA CAGTCTTCAC GAGCACATCG CTAATCTTGC AGGTAGCCCA |
| 2941 | GCTATCAAAA AGGGAATACT GCAGACCGTT AAGGTCGTGG ATGAACTCGT CAAAGTAATG |
| 3001 | GGAAGGCATA AGCCCGAGAA TATCGTTATC GAGATGGCCC GAGAGAACCA AACTACCCAG |
| 3061 | AAGGGACAGA AGAACAGTAG GGAAAGGATG AAGAGGATTG AAGAGGGTAT AAAAGAACTG |
| 3121 | GGGTCCCAAA TCCTTAAGGA ACACCCAGTT GAAAACACCC AGCTTCAGAA TGAGAAGCTC |
| 3181 | TACCTGTACT ACCTGCAGAA CGGCAGGGAC ATGTACGTGG ATCAGGAACT GGACATCAAT |
| 3241 | CGGCTCTCCG ACTACGACGT GGATCATATC GTGCCCCAGT CTTTTCTCAA AGATGATTCT |
| 3301 | ATTGATAATA AAGTGTTGAC AAGATCCGAT AAAAATAGAG GGAAGAGTGA TAACGTCCCC |
| 3361 | TCAGAAGAAG TTGTCAAGAA AATGAAAAAT TATTGGCGGC AGCTGCTGAA CGCCAAACTG |
| 3421 | ATCACACAAC GGAAGTTCGA TAATCTGACT AAGGCTGAAC GAGGTGGCCT GTCTGAGTTG |
| 3481 | GATAAAGCCG GCTTCATCAA AAGGCAGCTT GTTGAGACAC GCCAGATCAC CAAGCACGTG |
| 3541 | GCCCAAATTC TCGATTCACG CATGAACACC AAGTACGATG AAAATGACAA ACTGATTCGA |
| 3601 | GAGGTGAAAG TTATTACTCT GAAGTCTAAG CTGGTCTCAG ATTTCAGAAA GGACTTTCAG |
| 3661 | TTTTATAAGG TGAGAGAGAT CAACAATTAC CACCATGCGC ATGATGCCTA CCTGAATGCA |
| 3721 | GTGGTAGGCA CTGCACTTAT CAAAAAATAT CCCAAGCTTG AATCTGAATT TGTTTACGGA |
| 3781 | GACTATAAAG TGTACGATGT TAGGAAAATG ATCGCAAAGT CTGAGCAGGA AATAGGCAAG |
| 3841 | GCCACCGCTA AGTACTTCTT TTACAGCAAT ATTATGAATT TTTTCAAGAC CGAGATTACA |
| 3901 | CTGGCCAATG GAGAGATTCG GAAGCGACCA CTTATCGAAA CAAACGGAGA AACAGGAGAA |
| 3961 | ATCGTGTGGG ACAAGGGTAG GGATTTCGCG ACAGTCCGGA AGGTCCTGTC CATGCCGCAG |
| 4021 | GTGAACATCG TTAAAAAGAC CGAAGTACAG ACCGGAGGCT TCTCCAAGGA AAGTATCCTC |
| 4081 | CCGAAAAGGA ACAGCGACAA GCTGATCGCA CGCAAAAAAG ATTGGGACCC CAAGAAATAC |
| 4141 | GGCGGATTCG ATTCTCCTAC AGTCGCTTAC AGTGTACTGG TTGTGGCCAA AGTGGAGAAA |
| 4201 | GGGAAGTCTA AAAAACTCAA AAGCGTCAAG GAACTGCTGG GCATCACAAT CATGGAGCGA |
| 4261 | TCAAGCTTCG AAAAAAACCC CATCGACTTT CTCGAGGCGA AAGGATATAA AGAGGTCAAA |
| 4321 | AAAGACCTCA TCATTAAGCT TCCCAAGTAC TCTCTCTTTG AGCTTGAAAA CGGCCGGAAA |
| 4381 | CGAATGCTCG CTAGTGCGGG CGAGCTGCAG AAAGGTAACG AGCTGGCACT GCCCTCTAAA |
| 4441 | TACGTTAATT TCTTGTATCT GGCCAGCCAC TATGAAAAGC TCAAAGGGTC TCCCGAAGAT |
| 4501 | AATGAGCAGA AGCAGCTGTT CGTGGAACAA CACAAACACT ACCTTGATGA GATCATCGAG |
| 4561 | CAAATAAGCG AATTCTCCAA AAGAGTGATC CTCGCCGACG CTAACCTCGA TAAGGTGCTT |
| 4621 | TCTGCTTACA ATAAGCACAG GGATAAGCCC ATCAGGGAGC AGGCAGAAAA CATTATCCAC |
| 4681 | TTGTTTACTC TGACCAACTT GGGCGCGCCT GCAGCCTTCA AGTACTTCGA CACCACCATA |
| 4741 | GACAGAAAGC GGTACACCTC TACAAAGGAG GTCCTGGACG CCACACTGAT TCATCAGTCA |
| 4801 | ATTACGGGGC TCTATGAAAC AAGAATCGAC CTCTCTCAGC TCGGTGGAGA CAGCAGGGCT |
| 4861 | GACCCCAAGA AGAAGAGGAA GGTGTGAAAG GGTTCGATCC CTACCGGTTA GTAATGAGTT |
| 4921 | TAAACGGGGG AGGCTAACTG AAACACGGAA GGAGACAATA CCGGAAGGAA CCCGCGCTAT |
| 4981 | GACGGCAATA AAAAGACAGA ATAAAACGCA CGGGTGTTGG GTCGTTTGTT CATAAACGCG |
| 5041 | GGGTTCGGTC CCAGGGCTGG CACTCTGTCG ATACCCCACC GAGACCCCAT TGGGGCCAAT |
| 5101 | ACGCCCGCGT TTCTTCCTTT TCCCCACCCC ACCCCCCAAG TTCGGGTGAA GGCCCAGGGC |
| 5161 | TCGCAGCCAA CGTCGGGGCG GCAGGCCCTG CCATAGCAGA TCTGCGCAGC TGGGGCTCTA |
| 5221 | GGGGGTATCC CCACGCGCCC TGTAGCGGCG CATTAAGCGC GGCGGGTGTG GTGGTTACGC |
| 5281 | GCAGCGTGAC CGCTACACTT GCCAGCGCCC TAGCGCCCGC TCCTTTCGCT TTCTTCCCTT |
| 5341 | CCTTTCTCGC CACGTTCGCC GGCTTTCCCC GTCAAGCTCT AAATCGGGGC ATCCCTTTAG |
| 5401 | GGTTCCGATT TAGTGCTTTA CGGCACCTCG ACCCCAAAAA ACTTGATTAG GGTGATGGTT |
| 5461 | CACGTAGTGG GCCATCGCCC TGATAGACGG TTTTTCGCCC TTTGACGTTG GAGTCCACGT |
| 5521 | TCTTTAATAG TGGACTCTTG TTCCAAACTG GAACAACACT CAACCCTATC TCGGTCTATT |
| 5581 | CTTTTGATTT ATAAGGGATT TTGGGGATTT CGGCCTATTG GTTAAAAAAT GAGCTGATTT |
| 5641 | AACAAAAATT TAACGCGAAT TAATTCTGTG GAATGTGTGT CAGTTAGGGT GTGGAAAGTC |
| 5701 | CCCAGGCTCC CCAGCAGGCA GAAGTATGCA AAGCATGCAT CTCAATTAGT CAGCAACCAG |
| 5761 | GTGTGGAAAG TCCCCAGGCT CCCCAGCAGG CAGAAGTATG CAAAGCATGC ATCTCAATTA |
| 5821 | GTCAGCAACC ATAGTCCCGC CCCTAACTCC GCCCATCCCG CCCCTAACTC CGCCCAGTTC |
| 5881 | CGCCCATTCT CCGCCCCATG GCTGACTAAT TTTTTTTATT TATGCAGAGG CCGAGGCCGC |
| 5941 | CTCTGCCTCT GAGCTATTCC AGAAGTAGTG AGGAGGCTTT TTTGGAGGCC TAGGCTTTTG |
| 6001 | CAAAAAGCTC CCGGGAGCTT GTATATCCAT TTTCGGATCT GATCAAGAGA CAGGATGAGG |
| 6061 | ATCGTTTCGC ATGATTGAAC AAGATGGATT GCACGCAGGT TCTCCGGCCG CTTGGGTGGA |
| 6121 | GAGGCTATTC GGCTATGACT GGGCACAACA GACAATCGGC TGCTCTGATG CCGCCGTGTT |
| 6181 | CCGGCTGTCA GCGCAGGGGC GCCCGGTTCT TTTTGTCAAG ACCGACCTGT CCGGTGCCCT |
| 6241 | GAATGAACTG CAGGACGAGG CAGCGCGGCT ATCGTGGCTG GCCACGACGG GCGTTCCTTG |
| 6301 | CGCAGCTGTG CTCGACGTTG TCACTGAAGC GGGAAGGGAC TGGCTGCTAT TGGGCGAAGT |
| 6361 | GCCGGGGCAG GATCTCCTGT CATCTCACCT TGCTCCTGCC GAGAAAGTAT CCATCATGGC |
| 6421 | TGATGCAATG CGGCGGCTGC ATACGCTTGA TCCGGCTACC TGCCCATTCG ACCACCAAGC |
| 6481 | GAAACATCGC ATCGAGCGAG CACGTACTCG GATGGAAGCC GGTCTTGTCG ATCAGGATGA |
| 6541 | TCTGGACGAA GAGCATCAGG GGCTCGCGCC AGCCGAACTG TTCGCCAGGC TCAAGGCGCG |
| 6601 | CATGCCCGAC GGCGAGGATC TCGTCGTGAC CCATGGCGAT GCCTGCTTGC CGAATATCAT |
| 6661 | GGTGGAAAAT GGCCGCTTTT CTGGATTCAT CGACTGTGGC CGGCTGGGTG TGGCGGACCG |
| 6721 | CTATCAGGAC ATAGCGTTGG CTACCCGTGA TATTGCTGAA GAGCTTGGCG GCGAATGGGC |
| 6781 | TGACCGCTTC CTCGTGCTTT ACGGTATCGC CGCTCCCGAT TCGCAGCGCA TCGCCTTCTA |
| 6841 | TCGCCTTCTT GACGAGTTCT TCTGAGCGGG ACTCTGGGGT TCGCGAAATG ACCGACCAAG |
| 6901 | CGACGCCCAA CCTGCCATCA CGAGATTTCG ATTCCACCGC CGCCTTCTAT GAAAGGTTGG |
| 6961 | GCTTCGGAAT CGTTTTCCGG GACGCCGGCT GGATGATCCT CCAGCGCGGG GATCTCATGC |
| 7021 | TGGAGTTCTT CGCCCACCCC AACTTGTTTA TTGCAGCTTA TAATGGTTAC AAATAAAGCA |
| 7081 | ATAGCATCAC AAATTTCACA AATAAAGCAT TTTTTTCACT GCATTCTAGT TGTGGTTTGT |
| 7141 | CCAAACTCAT CAATGTATCT TATCATGTCT GTATACCGTC GACCTCTAGC TAGAGCTTGG |
| 7201 | CGTAATCATG GTCATAGCTG TTTCCTGTGT GAAATTGTTA TCCGCTCACA ATTCCACACA |
| 7261 | ACATACGAGC CGGAAGCATA AAGTGTAAAG CCTGGGGTGC CTAATGAGTG AGCTAACTCA |
| 7321 | CATTAATTGC GTTGCGCTCA CTGCCCGCTT TCCAGTCGGG AAACCTGTCG TGCCAGCTGC |
| 7381 | ATTAATGAAT CGGCCAACGC GCGGGGAGAG GCGGTTTGCG TATTGGGCGC TCTTCCGCTT |
| 7441 | CCTCGCTCAC TGACTCGCTG CGCTCGGTCG TTCGGCTGCG GCGAGCGGTA TCAGCTCACT |
| 7501 | CAAAGGCGGT AATACGGTTA TCCACAGAAT CAGGGGATAA CGCAGGAAAG AACATGTGAG |
| 7561 | CAAAAGGCCA GCAAAAGGCC AGGAACCGTA AAAAGGCCGC GTTGCTGGCG TTTTTCCATA |
| 7621 | GGCTCCGCCC CCCTGACGAG CATCACAAAA ATCGACGCTC AAGTCAGAGG TGGCGAAACC |
| 7681 | CGACAGGACT ATAAAGATAC CAGGCGTTTC CCCCTGGAAG CTCCCTCGTG CGCTCTCCTG |
| 7741 | TTCCGACCCT GCCGCTTACC GGATACCTGT CCGCCTTTCT CCCTTCGGGA AGCGTGGCGC |
| 7801 | TTTCTCAATG CTCACGCTGT AGGTATCTCA GTTCGGTGTA GGTCGTTCGC TCCAAGCTGG |
| 7861 | GCTGTGTGCA CGAACCCCCC GTTCAGCCCG ACCGCTGCGC CTTATCCGGT AACTATCGTC |
| 7921 | TTGAGTCCAA CCCGGTAAGA CACGACTTAT CGCCACTGGC AGCAGCCACT GGTAACAGGA |
| 7981 | TTAGCAGAGC GAGGTATGTA GGCGGTGCTA CAGAGTTCTT GAAGTGGTGG CCTAACTACG |
| 8041 | GCTACACTAG AAGGACAGTA TTTGGTATCT GCGCTCTGCT GAAGCCAGTT ACCTTCGGAA |
| 8101 | AAAGAGTTGG TAGCTCTTGA TCCGGCAAAC AAACCACCGC TGGTAGCGGT GGTTTTTTTG |
| 8161 | TTTGCAAGCA GCAGATTACG CGCAGAAAAA AAGGATCTCA AGAAGATCCT TTGATCTTTT |
| 8221 | CTACGGGGTC TGACGCTCAG TGGAACGAAA ACTCACGTTA AGGGATTTTG GTCATGAGAT |
| 8281 | TATCAAAAAG GATCTTCACC TAGATCCTTT TAAATTAAAA ATGAAGTTTT AAATCAATCT |
| 8341 | AAAGTATATA TGAGTAAACT TGGTCTGACA GTTACCAATG CTTAATCAGT GAGGCACCTA |
| 8401 | TCTCAGCGAT CTGTCTATTT CGTTCATCCA TAGTTGCCTG ACTCCCCGTC GTGTAGATAA |
| 8461 | CTACGATACG GGAGGGCTTA CCATCTGGCC CCAGTGCTGC AATGATACCG CGAGACCCAC |
| 8521 | GCTCACCGGC TCCAGATTTA TCAGCAATAA ACCAGCCAGC CGGAAGGGCC GAGCGCAGAA |
| 8581 | GTGGTCCTGC AACTTTATCC GCCTCCATCC AGTCTATTAA TTGTTGCCGG GAAGCTAGAG |
| 8641 | TAAGTAGTTC GCCAGTTAAT AGTTTGCGCA ACGTTGTTGC CATTGCTACA GGCATCGTGG |
| 8701 | TGTCACGCTC GTCGTTTGGT ATGGCTTCAT TCAGCTCCGG TTCCCAACGA TCAAGGCGAG |
| 8761 | TTACATGATC CCCCATGTTG TGCAAAAAAG CGGTTAGCTC CTTCGGTCCT CCGATCGTTG |
| 8821 | TCAGAAGTAA GTTGGCCGCA GTGTTATCAC TCATGGTTAT GGCAGCACTG CATAATTCTC |
| 8881 | TTACTGTCAT GCCATCCGTA AGATGCTTTT CTGTGACTGG TGAGTACTCA ACCAAGTCAT |
| 8941 | TCTGAGAATA GTGTATGCGG CGACCGAGTT GCTCTTGCCC GGCGTCAATA CGGGATAATA |
| 9001 | CCGCGCCACA TAGCAGAACT TTAAAAGTGC TCATCATTGG AAAACGTTCT TCGGGGCGAA |
| 9061 | AACTCTCAAG GATCTTACCG CTGTTGAGAT CCAGTTCGAT GTAACCCACT CGTGCACCCA |
| 9121 | ACTGATCTTC AGCATCTTTT ACTTTCACCA GCGTTTCTGG GTGAGCAAAA ACAGGAAGGC |
| 9181 | AAAATGCCGC AAAAAAGGGA ATAAGGGCGA CACGGAAATG TTGAATACTC ATACTCTTCC |
| 9241 | TTTTTCAATA TTATTGAAGC ATTTATCAGG GTTATTGTCT CATGAGCGGA TACATATTTG |
| 9301 | AATGTATTTA GAAAAATAAA CAAATAGGGG TTCCGCGCAC ATTTCCCCGA AAAGTGCCAC |
| 9361 | CTGACGTCGA CGGATCGGGA GATCTCCCGA TCCCCTATGG TCGACTCTCA GTACAATCTG |
| 9421 | CTCTGATGCC GCATAGTTAA GCCAGTATCT GCTCCCTGCT TGTGTGTTGG AGGTCGCTGA |
| 9481 | GTAGTGCGCG AGCAAAATTT AAGCTACAAC AAGGCAAGGC TTGACCGACA ATTGCATGAA |
| 9541 | GAATCTGCTT AGG |

Single cell clones were then generated by clonal selection (Richter et al., 2012), splitted, propagated and screened by a qPCR-based loss-of-wild-type allele approach (Vochozkova et al., 2019). The qPCR was performed using primers shown in Table 1 (SEQ ID NOs: 8-13). The results are shown in FIG. 8. The cell clones that appeared to show a reduced number of porcine WT alleles from 2 to 1 were further re-examined using end-point PCR Sanger sequencing for the abundance of a correct porcine WT allele and the abundance of the floxed human PLN WT and the mutated PLN R14del copies of exon 2.

In parallel with the generation and screening of SCCs for SCNT, the rescue cassette removal protocol was tested and optimised for a limited cellular material prior to the generation of the founder pigs. As a considerable loss of cells during nucleofection of small cell numbers was obvious in all experiments and the consequences of nucleofection on the developmental potential of SCC in embryos were difficult to estimate, the PLN SCCs were prepared and verified for first SCNT experiments either by lipofection with Cre-mRNA or nucleofection with a Cre-encoding plasmid.

In total, 4 SCNT experiments with composites of lipofected and nucleofected SCCs have been conducted, resulting in 2 pregnancies, yielding 2 and 3 piglets, respectively. One of piglets was stillborn, two died shortly after birth, but a further 2 animals survived, in accordance with the long-term outcome in SCNT experiments. The surviving animals were raised and have recently been subjected to the mating process for establishing a breeding herd. The genetic constellations of the piglets were analysed by PCR. Sequences of primer pairs 1-6 are shown in Table 1 (SEQ ID NOs: 14-25). The stillborn piglet and one of the dead animals lacked the rescue cassette while the others, including the viable animals retained the rescue cassette, as shown in FIGS. 9 and 10.

In a number of additional attempts, primary cells isolated from the surviving founder animals were obtained and used for 2 further SCNT experiments. These resulted in a single pregnancy yielding 5 re-cloned piglets, all of them lacking the rescue cassette, as shown in FIG. 9. Two of these re-cloned piglets have survived and have been raised for phenotypical examination and, if possible, reproduction, according the strategic options shown in FIG. 6.

### Phenotype Characterization

Considering the very specific characteristics of phenotype development in cardiomyopathy patients carrying mutated PLN, a “reverse translational” approach was followed for the characterization of the transgenic PLN pig model. The approach involves adaptation of diagnostic methods from the patients to the disease model, being the PLN pig. This specifically includes electrocardiogram (ECG) measurements aiming at detecting typical arrhythmias, T-wave inversion and evaluation of the QRS complex. Importantly echocardiography is performed for standard parameters such as ejection fraction, fractional shortening, end diastolic volume or cardiac output. In addition, upon the appearance of early PLN-mutation phenotype-specific signs of ventricular walls dilation, post-systolic shortening of the left ventricular apex is performed according to clinical procedures.

This initial end-point characterization was performed in a single R14del re-cloned animal (no. #12436, genetic constellation shown in FIG. 9), which for that reason was determined to be sacrificed at an age of 6 months. A WT pig and a PLN transgenic pig carrying the rescue cassette, were used as controls. Expression analysis by PCR confirmed the correct splicing of the respective genetic constellations, as shown in FIG. 10. Primer pairs 1-6, as indicated in FIGS. 9 and 10 are shown in Table 1 (SEQ ID NOs: 14-25).

At the quantitative level as measured by RT-PCR (primers shown in Table 1, SEQ ID NOs: 26-29), it appeared that the humanized alleles show lower abundance levels as compared with the porcine alleles, as shown in FIGS. 11-12. Albeit an influence of epigenetic regulation in the cloned animals cannot be excluded at the moment, the data may be indicating the potential relevance of sequence-specific differences present between porcine and the humanized 3′-UTRs. This hypothesis appears to be in line with in silico data shown in FIG. 13, which appears to be suggesting that the human and the porcine 3′-UTRs of the PLN transcripts may have distinct regulatory properties, fitting to a multi-species-based prediction model of regulatory properties (Grotz et al., 2022).

Last but not least, to verify if the PLN transgenic pig model is successful at recreating the diseased heart phenotype of the human subjects suffering from the PLN-associated cardiomyopathy, a series of histological assessments was performed on the heart tissue obtained from the R14del re-cloned animal #12436 and compared to a WT age-matched control animal.

Already at this early state, a staining pattern suggestive of a formation of fibrotic strains and a compacted heart structure was observed and is shown in FIG. 14. The latter observation further correlated with an increased heart: body weight ratio as determined at the timepoint of sampling.

Then, immunohistochemical staining of human PLN was performed on the heart tissue sections obtained from the R14del re-cloned animal #12436 (poR14del), the age-matched WT control animal (poWT), and a human staining control tissue (hu) and is shown in FIG. 15. The anti-PLN staining confirmed the protein's intracellular localization across the sarcoplasmic reticulum. Interestingly, the staining also showed a specific to the R14del animal tissues, but not to control tissues, increased PLN protein staining in the peri-nuclear space of the cardiomyocytes, suggesting the protein's accumulation to that space. This increased PLN-associated staining is indicated by arrows in FIG. 15, and is similar to the formation of PLN aggregates that are seen in human PLN patients (Te Rijdt et al., 2017).

The above data is extremely promising and shows that even at this early stage of development, the generated herein transgenic model of human cardiomyopathy is developing a phenotype that is reminiscent of the human disease. Consequently, it can be concluded that the new model generation appears to have been successful.

### Confirmation of the Phenotype in F1 Generation Animals

In a cohort of 6 months old F1 generation animals (n=4 R14del animals, n=6 WT controls among which n=3 were siblings of the R14del animals and n=3 were more distantly related but from the same breed), electrophysiological cardiac mapping was conducted in a clinically relevant setting (Rythmia, Boston Scientific). The results are shown in FIG. 16. Notably, they demonstrate evident electrophysiological malformations (indicated by arrows in panel B) present solely in all of the R14del animals but not in the WT control animals, which are reminiscent of the split potentials routinely observed in the human patients carrying the R14del mutation. The interpretation of the results was performed by an experienced practician routinely performing electrophysiology examination of human and pig hearts in pre-clinical studies, who has confirmed that the observed malformations constituted a unique signature of the R14del animals and cannot be observed in a healthy pig heart. These results ultimately show that the human heart disease phenotype associated with the mutation has successfully been reproduced in the presented herein transgenic animal disease model.

The animals were sacrificed immediately after the electrophysiological mapping and their hearts were sampled. The hearts were then cut as complete slices, fixed in PFA and stained for fibrotic depositions with Sirius Red according to the manufacturer's recommendations. The results are shown in FIG. 17 and demonstrate that the electrophysiological malformations observed in the R14del animals in the cardiac mapping correlated with the presence of increased fatty-fibrotic deposition observed at the border of the myocard and, specifically in the interstitium (panel A). While fibrotic tissue in the WT control animals appears at the border zones of the myocard or around the vessels (B), the extracellular matrix deposition in the R14del animals was visibly increased and appeared to be growing into the interstitial tissue. These results confirm the successful reproduction of the disease phenotype and suitability of the presented herein animal disease model for the purpose of conducting further translational research in the benefit of human patients suffering from cardiomyopathic disorders.

### REFERENCES

Chien, K. R., Ross, J., Jr., and Hoshijima, M. (2003). Calcium and heart failure: the cycle game. Nat Med 9, 508-509.
Dave J. at al. (2022). Gene editing reverses arrhythmia susceptibility in humanized PLN-R14del mice: modelling a European cardiomyopathy with global impact. Cardiovascular research, vol. 118, no. 15
Doevendans P., Glijnis P., Kranias E. (2019). “Leducq Transatlantic Network of Excellence to Cure Phospholamban-Induced Cardiomyopathy (CURE-PLAN)”, Circulation Res., vol. 125, no. 7.
Eijgenraam, T. R., Boukens, B. J., Boogerd, C. J., Schouten, E. M., van de Kolk, C. W. A., Stege, N. M., Te Rijdt, W. P., Hoorntje, E. T., van der Zwaag, P. A., van Rooij, E., et al. (2020). The phospholamban p. (Arg14del) pathogenic variant leads to cardiomyopathy with heart failure and is unreponsive to standard heart failure therapy. Sci Rep 10, 9819.
Glaves, J. P., Primeau J. O., Espinoza-Fonseca, L. M., Lemieux, M. J., Young, H. S, (2019). The Phospholamban Pentamer Alters Function of the Sarcoplasmic Reticulum Calcium Pump SERCA. Biophys J. DOI: 10.1016/j.bpj.2019.01.013
Gorski, P. A., Trieber, C. A., Ashrafi, G, Young, H. S. (2015). Regulation of the sarcoplasmic reticulum calcium pump by divergent phospholamban isoforms in zebrafish. J Biol Chem. DOI: 10.1074/jbc.M114.585604
Grotz, S., Schafer, J., Wunderlich, K. A., Ellederova, Z., Auch, H., Bahr, A., Runa-Vochozkova, P., Fadl, J., Arnold, V., Ardan, T., et al. (2022). Early disruption of photoreceptor cell architecture and loss of vision in a humanized pig model of usher syndromes. EMBO Mol Med 14, e14817.
Haghighi K. et al. (2006): A mutation in the human phospholamban gene, deleting arginine 14, results in lethal, hereditary cardiomyopathy”, PNAS, vol. 103, no. 5, 31 Jan. 2006 (2006-01-31), pages 1388-1393, XP093081439, ISSN: 0027-8424, DOI: 10.1073/pnas.0510519103.
Haghighi K. et al. (2013): The human phospholamban Argi 4-deletion mutant localizes to plasma membrane and interacts with the Na/K-ATPase, J. of Molecular and Cellular Cardiology, vol. 52, no. 3, pages 773-782, XP093081438, GB ISSN: 0022-2828, DOI: 10.1016/j.yjmcc.2011.11.012.
Hof, I. E., van der Heijden, J. F., Kranias, E. G., Sanoudou, D., de Boer, R. A., van Tintelen, J. P., van der Zwaag, P. A., and Doevendans, P. A. (2019). Prevalence and cardiac phenotype of patients with a phospholamban mutation. Neth Heart J 27, 64-69.
Joseph, P.; Leong, D.; McKee, M.; Anand, S. S.; Schwalm, J.-D.; Teo, K.; Mente, A.; Yusuf, S. Reducing the Global Burden of Cardiovascular Disease, Part 1: The Epidemiology and Risk Factors. Circ. Res. 2017, 121, 677-694.
Kurome, M., Kessler, B., Wuensch, A., Nagashima, H., and Wolf, E. (2015). Nuclear transfer and transgenesis in the pig. Methods Mol Biol 1222, 37-59.
Primeau 2018 is DOI: 10.1007/978-981-10-7757-9_8,
Raad, N., Bittihn, P., Cacheux, M., Jeong, D., Ilkan, Z., Ceholski, D., Kohlbrenner, E., Zhang, L., Cai, C. L., Kranias, E. G., et al. (2021). Arrhythmia Mechanism and Dynamics in a Humanized Mouse Model of Inherited Cardiomyopathy Caused by Phospholamban R14del Mutation. Circulation 144, 441-454.
Richter, A., Kurome, M., Kessler, B., Zakhartchenko, V., Klymiuk, N., Nagashima, H., Wolf, E., and Wuensch, A. (2012). Potential of primary kidney cells for somatic cell nuclear transfer mediated transgenesis in pig. BMC Biotechnol 12, 84.
Rogalska M. et al. (2023). Isoform changes of action potential regulators in the ventricles of arrhythmogenic phospholamban-R14del humanized mouse hearts. Metabolism Clinical and Experimental, vol. 138, DOI: 10.1016/j.metabol.2022.155344
Seidel, K., Andronesi, O. C., Krebs, J., Griesinger, C., Young, H. S., Becker, S, and Baldus, M. (2008). Structural characterization of Ca(2+)-ATPase-bound phospholamban in lipid bilayers by solid-state nuclear magnetic resonance (NMR) spectroscopy. DOI: 10.1021/bi7024194
Te Rijdt, W. P., van der Klooster, Z. J., Hoorntje, E. T., Jongbloed, J. D. H., van der Zwaag, P. A., Asselbergs, F. W., Dooijes, D., de Boer, R. A., van Tintelen, J. P., van den Berg, M. P., et al. (2017). Phospholamban immunostaining is a highly sensitive and specific method for diagnosing phospholamban p.Arg 14del cardiomyopathy. Cardiovasc Pathol 30, 23-26.
van Tintelen, J. P.; Wilde, A. A. M.; Jongbloed, J. D. H. Recurrent and founder mutations in inherited cardiac diseases in the Netherlands. Neth. Hear. J. 2009, 17, 407-408.
Vafiadaki E. et al. (2023) Phosphoiamban R14del disease: The past, the present and the future, Frontiers in Cardiovascular Medicine., vol. 10, DOI: 10.3389/fcvm.2023.1162205
Vochozkova, P., Simmet, K., Jemiller, E. M., Wunsch, A., and Klymiuk, N. (2019). Gene Editing in Primary Cells of Cattle and Pig. Methods Mol Biol 1961, 271-289.
Warming, S., Costantino, N., Court, D. L., Jenkins, N. A., and Copeland, N. G. (2005). Simple and highly efficient BAC recombineering using galK selection. Nucleic Acids Res 33, e36.

### SUPPLEMENTARY INFORMATION ON THE SEQUENCES SHOWN IN THE FIGURES

For completeness, the sequences displayed in several of the Figures as indicated below, are herewith explained in the Table underneath with the reference to their SEQ ID NOs. as provided in the accompanying electronic sequence listing file compliant with the ST.26 standard.

| Sequence | | | Comments explaining the sequence (for |
|---|---|---|---|
| location | # | Sequence | synthetic construct sequences in ST.26 file |
| FIG. 2 | SEQ ID | cf. SEQ ID NO: 1 as contained | Human PLN protein (FL, WT, RefSeq no. |
| | NO: 1 | in the text and sequence | NP_002658.1) |
| | | listing as filed | |
| | SEQ ID | ILLLAACISFVLAWFEEGEETITAFVEP | Human ATP2A2; NCBI Reference Sequence: |
| | NO: 33 | FVILLILVANAIVGVWQERNAENAIEAL | NP_001672.1 shown in FIG.3 from I64 till |
| | | KEYEPEMGKVYRQDRKSVQRIKAKDIVP | N817 |
| | | GDIVEIAVGDKVPADIRLTSIKSTTLRV | |
| | | DQSILTGESVSVIKHTDPVPDPRAVNQD | |
| | | KKNMLFSGTNIAAGKAMGVVVATGVNTE | |
| | | IGKIRDEMVATEQERTPLQQKLDEFGEQ | |
| | | LSKVISLICIAVWIINIGHFNDPVHGGS | |
| | | WIRGAIYYFKIAVALAVAAIPEGLPAVI | |
| | | TTCLALGTRRMAKKNAIVRSLPSVETLG | |
| | | CTSVICSDKTGTLTTNQMSVCRMFILDR | |
| | | VEGDTCSLNEFTITGSTYAPIGEVHKDD | |
| | | KPVNCHQYDGLVELATICALCNDSALDY | |
| | | NEAKGVYEKVGEATETALTCLVEKMNVF | |
| | | DTELKGLSKIERANACNSVIKQLMKKEF | |
| | | TLEFSRDRKSMSVYCTPNKPSRTSMSKM | |
| | | FVKGAPEGVIDRCTHIRVGSTKVPMTSG | |
| | | VKQKIMSVIREWGSGSDTLRCLALATHD | |
| | | NPLRREEMHLEDSANFIKYETNLTFVGC | |
| | | VGMLDPPRIEVASSVKLCRQAGIRVIMI | |
| | | TGDNKGTAVAICRRIGIFGQDEDVTSKA | |
| | | FTGREFDELNPSAQRDACLNARCFARVE | |
| | | PSHKSKIVEFLQSFDEITAMTGDGVNDA | |
| | | PALKKAEIGIAMGSGTAVAKTASEMVLA | |
| | | DDNFSTIVAAVEEGRAIYNNMKQFIRYL | |
| | | ISSNVGEVVCIFLTAALGFPEALIPVQL | |
| | | LWVNLVTDGLPATALGENPPDLDIMN | |
| FIG. 3 | SEQ ID | CLLLICIIVMLL | Human PLN fragment labelled in FIG. 3 as |
| | NO: 34 | | Gorski 2015, Seidel 2008 (including human |
| | | | PLN C46-M50) |
| | SEQ ID | PQQARQKLQNLFINFCL | Human PLN fragment labelled in FIG. 3 as |
| | NO: 35 | | Gorski 2015, Glaves 2019 and as Seidel 2008 |
| | SEQ ID | KLQNLFINFCLILICLLLICII | Human PLN fragment labelled in FIG. 3 as |
| | NO: 36 | | Glaves 2019 (including human PLN Q29-145) |
| | SEQ ID | PQQARQKLQNL | Human PLN fragment labelled in FIG. 3 as |
| | NO: 37 | | Gorski 2015, Primeau 2018 (including human |
| | | | PLN R25-K27) |
| | SEQ ID | MEKVQYL | Human PLN fragment labelled in FIG. 3 as |
| | NO: 38 | | Seidel 2008 (including human PLN E2-K3) |
| FIG. 7 | SEQ ID | TNAATTTTNNTTATAGAAATTTINGGAT | 1st chromatogram sequence from the top in |
| | NO: 39 | CCTTGGGGTCTTTATTGAGAAGTTGG | FIG. 7 |
| | | | (#1)-sequenced fragment of the targeting |
| | | | vector |
| | SEQ ID | TACCTCACTCGCTCAGCTATAAGANCAN | 2nd chromatogram sequence from the top in |
| | NO: 40 | CCTCNANTGTTGTGATGCCTCAACNA | FIG. 7 (#2)-sequenced fragment of the |
| | | | targeting vector |
| | SEQ ID | AATTAAAGCCAGAACAGGGTACACTAGC | 3rd chromatogram sequence from the top in |
| | NO: 41 | TNGANTGCNNGNCGNCNNAATTCTAC | FIG. 7 (#3)-sequenced fragment of the |
| | | | targeting vector |
| | SEQ ID | AAAAATTAAAGCCAGAACAGGGTACACT | 4th chromatogram sequence from the top in |
| | NO: 42 | AGCTTGGCTGCAGGTCGTCGAAATTCT | FIG. 7 (#4)-sequenced fragment of the |
| | | | targeting vector |
| | SEQ ID | GAACCCTTAATATAACTTCGTATAATGT | 5th chromatogram sequence from the top in |
| | NO: 43 | ATGCTATACGAAGTTAT GGATCCTTGG | FIG. 7 (#5)-sequenced fragment of the |
| | | | targeting vector |
| FIG. 10 | SEQ ID | ccctaaactaaaatgcatgacaataaac | 1st chromatogram sequence from the top in |
| | NO: 44 | gctagctggcaATAACTTCGTATAATGT | FIG. 10 (fragment of SEQ ID NO. 30) |
| | | ATGCTATACGAAGTTATggatccttggg | |
| | | gtcttt | |
| | SEQ ID | ccctaaactaaaatgcatgacaataaac | 2nd chromatogram sequence from the top in |
| | NO: 45 | gctagctggcaATAACTTCGTATAATGT | FIG. 10 (fragment of SEQ ID NO. 30) |
| | | ATGCTATACGAAGTTATgggatccttgg | |
| | | ggtcttt | |
| | SEQ ID | CCAATACCTCACTCGCTCAGCTATAAGA | 3rd chromatogram sequence from the top in |
| | NO: 46 | agaGCCTCAACCATTGAAATGCCTCAAC | FIG. 10 and labelled as R14 (fragment of |
| | | AAGCACGTCAA | SEQ ID NO. 30) |
| | SEQ ID | CCAATACCTCACTCGCTCAGCTATAAGA | 3rd & 4th chromatogram sequence from the |
| | NO: 47 | -GCCTCAACCATTGAAATGCCTCAACAA | top in FIG. 10 and labelled as 14del |
| | | GCACGTCAAAAG | (fragment of SEQ ID NO. 30) |
| | SEQ ID | QYLTRSAIRRASTIEMPQQARQ | translation of chromatogram sequence |
| | NO: 48 | | labelled as R14 in FIG. 10 (fragment of SEQ |
| | | | ID NO: 1) |
| | SEQ ID | QYLTRSAIRASTIEMPQQARQK | translation of chromatogram sequence |
| | NO: 49 | | labelled as 14del in FIG. 10 (fragment of |
| | | | SEQ ID NO: 2) |
| | SEQ ID | aaaattaaagccagaacagggtacacta | 5th chromatogram sequence from the top in |
| | NO: 50 | gcttggctgcaggtcgtcgaaattcta | FIG. 10 (fragment of SEQ ID NO. 30) |
| | SEQ ID | gcttgcggaacccttaatATAACTTC | 6th chromatogram sequence from the top in |
| | NO: 51 | GTATAATGTATGCTATACGAAGTTATgg | FIG. 10 (fragment of SEQ ID NO. 30) |
| | | atccttggggtctttattgaga | |
| FIG. 11 | SEQ ID | CGCTCTGCTATTAGAAGAGCTTCAACCA | Chromatogram fragment shown next to label |
| | NO: 52 | TTCAAAT | ″po WT″ in FIG. 11 |
| | SEQ ID | CGCTCAGCTATAAGAAGAGCCTCAACCA | Chromatogram fragment shown next to label |
| | NO: 53 | TTGAAAT | ″po rescue″ in FIG. 11 |
| | SEQ ID | CGCTCAGCTATAAGAGCCTCAACCATTC | Chromatogram fragment shown next to label |
| | NO: 54 | AAAT | ″po R14del″ in FIG. 11 |

1. A transgenic ungulate comprises:
a genome comprising at least one copy of a first transgene encoding for at least a first part of a human phospholamban (PLN) protein.

2. The transgenic ungulate according to claim 1, wherein expression of the first transgene results in production of the first part of the human PLN protein being at least a part of a human PLN R14del mutant protein comprising at least the PLN protein R14del mutation.

3. The transgenic ungulate according to claim 1, wherein the first part of a human PLN protein is a full length (FL) human PLN R14del mutant protein represented by SEQ ID NO: 2.

4. The transgenic ungulate according to claim 1, wherein the first transgene comprises a first DNA segment comprising a human PLN gene mutant exon 2 encoding for the FL human PLN R14del mutant protein, optionally wherein the first DNA segment further comprises a human PLN gene exon 1 or a part thereof.

5. The transgenic ungulate according to claim 1, wherein the first transgene further comprises at least one SNP selected from rs12198461 and rs1051429.

6. The transgenic ungulate according to claim 1, wherein the expression of the first transgene is inducible.

7. The transgenic ungulate according to claim 1, wherein the genome further comprises at least one copy of a second transgene encoding for at least a part of a wild-type (WT) human PLN protein, wherein expression of the second transgene results in production of the part of a WT human PLN protein.

8. The transgenic ungulate according to claim 7, wherein the second transgene is comprised as part of a rescue cassette.

9. The transgenic ungulate according to claim 1, wherein at least the first transgene is knocked-in into the locus of native PLN gene.

10. The transgenic ungulate according to claim 1, being a transgenic livestock ungulate.

11. The transgenic ungulate according to claim 1, obtained or obtainable by a method comprising the steps of:
(a) genetic modification of primary ungulate cells;
(b) verification of single cell clones genetically modified in (a) for the presence of a signal that can be correlated with the at least first transgene and/or the second transgene; and
(c) use of the single cell clones selected following the verification in (b) for somatic cell nuclear transfer.

12. The transgenic ungulate obtained or obtainable following the method of claim 11, wherein the method further comprises the step of:
inducing the expression of the first transgene by removing the removable rescue cassette comprising the second transgene either
in vitro, preferably in primary ungulate cells, more preferably by a vector transfection or viral vector infection or by micro-injection of means of removing the removable rescue cassette, preferably Cre protein or Cre-encoding mRNA, most preferably into a zygote carrying the removable rescue cassette; or
in vivo by performing a cross with a second transgenic ungulate comprising a second genome comprising a third transgene encoding for means adapted to remove the removable rescue cassette following the third transgene's expression.

13. A progeny of the transgenic ungulate obtained or obtainable following the method according to claim 11, wherein the genome of the progeny comprises at least the first transgene or the second transgene.

14. An organ isolated from the transgenic ungulate or a progeny of the transgenic ungulate according to claim 11.

15. A cell isolated from the transgenic ungulate or a progeny of the transgenic ungulate, or an organ isolated from the transgenic ungulate according to claim 11.

16. A method for translational research and/or as a human heart disease model system, said method comprising the step of:
employing at least one transgenic ungulate, or a progeny of the transgenic ungulate, or an organ isolated from the transgenic ungulate, or an isolated cell from the transgenic ungulate according to claim 11.

17. A method of screening biologically active agents for identification of a therapeutic agent for use in the treatment of a human heart disease, the method comprising at least the steps of:
administration of at least one biologically active agent at least once either in vivo to the transgenic ungulate, or a progeny of the transgenic ungulate, or in vitro into an organ isolated from the transgenic ungulate or an isolated cell from the transgenic ungulate according to claim 11.
